SEQUENCE LISTING (5 sequences)

SEQ ID NO: 1 - RNA, 2124 nt, Homo sapiens
  source 1..2124: mol_type = mRNA
atgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactag

SEQ ID NO: 2 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 3 - RNA, 1822 nt, Homo sapiens
  source 1..1822: mol_type = mRNA
agaaaggaacacagtaaactgaattgatccgtttagaagtttacaatgaagtttcttctaatactgctcctgcaggccactgcttctggagctcttcccctgaacagctctacaagcctggaaaaaaataatgtgctatttggtgaaagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatgtccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggccgtaatgttccccacctacaaatatgttgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctactctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaaggccaagagaattctatttgaagcatgctctgtaagttgcttcctaacatccttggactgagaaattatacttacttctggcataactaaaattaagtatatatattttggctcaaataaaattgaaaaaaaaa

SEQ ID NO: 4 - protein, 470 aa, Homo sapiens
MKFLLILLLQATASGALPLNSSTSLEKNNVLFGERYLEKFYGLEINKLPVTKMKYSGNLMKEKIQEMQHFLGLKVTGQLDTSTLEMMHAPRCGVPDVHHFREMPGGPVWRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNPDNSEPALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 5 - RNA, 2124 nt, synthetic construct
  source 1..2124: mol_type = other RNA
  modified_base 2: mod_base = mo5u
  modified_base 4: mod_base = mo5u
  modified_base 8: mod_base = mo5u
  modified_base 10: mod_base = mo5u
  modified_base 20: mod_base = mo5u
  modified_base 23: mod_base = mo5u
  modified_base 26: mod_base = mo5u
  modified_base 29: mod_base = mo5u
  modified_base 32: mod_base = mo5u
  modified_base 35: mod_base = mo5u
  modified_base 38: mod_base = mo5u
  modified_base 41: mod_base = mo5u
  modified_base 46: mod_base = mo5u
  modified_base 49: mod_base = mo5u
  modified_base 52: mod_base = mo5u
  modified_base 53: mod_base = mo5u
  modified_base 83: mod_base = mo5u
  modified_base 86: mod_base = mo5u
  modified_base 89: mod_base = mo5u
  modified_base 91: mod_base = mo5u
  modified_base 92: mod_base = mo5u
  modified_base 102: mod_base = mo5u
  modified_base 104: mod_base = mo5u
  modified_base 116: mod_base = mo5u
  modified_base 131: mod_base = mo5u
  modified_base 142: mod_base = mo5u
  modified_base 146: mod_base = mo5u
  modified_base 148: mod_base = mo5u
  modified_base 154: mod_base = mo5u
  modified_base 160: mod_base = mo5u
  modified_base 170: mod_base = mo5u
  modified_base 179: mod_base = mo5u
  modified_base 190: mod_base = mo5u
  modified_base 200: mod_base = mo5u
  modified_base 212: mod_base = mo5u
  modified_base 215: mod_base = mo5u
  modified_base 218: mod_base = mo5u
  modified_base 221: mod_base = mo5u
  modified_base 233: mod_base = mo5u
  modified_base 235: mod_base = mo5u
  modified_base 239: mod_base = mo5u
  modified_base 257: mod_base = mo5u
  modified_base 272: mod_base = mo5u
  modified_base 281: mod_base = mo5u
  modified_base 295: mod_base = mo5u
  modified_base 302: mod_base = mo5u
  modified_base 311: mod_base = mo5u
  modified_base 319: mod_base = mo5u
  modified_base 320: mod_base = mo5u
  modified_base 328: mod_base = mo5u
  modified_base 329: mod_base = mo5u
  modified_base 341: mod_base = mo5u
  modified_base 346: mod_base = mo5u
  modified_base 362: mod_base = mo5u
  modified_base 367: mod_base = mo5u
  modified_base 370: mod_base = mo5u
  modified_base 374: mod_base = mo5u
  modified_base 382: mod_base = mo5u
  modified_base 385: mod_base = mo5u
  modified_base 393: mod_base = mo5u
  modified_base 395: mod_base = mo5u
  modified_base 407: mod_base = mo5u
  modified_base 410: mod_base = mo5u
  modified_base 421: mod_base = mo5u
  modified_base 422: mod_base = mo5u
  modified_base 433: mod_base = mo5u
  modified_base 434: mod_base = mo5u
  modified_base 440: mod_base = mo5u
  modified_base 442: mod_base = mo5u
  modified_base 452: mod_base = mo5u
  modified_base 461: mod_base = mo5u
  modified_base 466: mod_base = mo5u
  modified_base 467: mod_base = mo5u
  modified_base 476: mod_base = mo5u
  modified_base 478: mod_base = mo5u
  modified_base 481: mod_base = mo5u
  modified_base 497: mod_base = mo5u
  modified_base 503: mod_base = mo5u
  modified_base 508: mod_base = mo5u
  modified_base 509: mod_base = mo5u
  modified_base 515: mod_base = mo5u
  modified_base 535: mod_base = mo5u
  modified_base 541: mod_base = mo5u
  modified_base 542: mod_base = mo5u
  modified_base 560: mod_base = mo5u
  modified_base 563: mod_base = mo5u
  modified_base 574: mod_base = mo5u
  modified_base 575: mod_base = mo5u
  modified_base 593: mod_base = mo5u
  modified_base 610: mod_base = mo5u
  modified_base 611: mod_base = mo5u
  modified_base 626: mod_base = mo5u
  modified_base 628: mod_base = mo5u
  modified_base 635: mod_base = mo5u
  modified_base 647: mod_base = mo5u
  modified_base 650: mod_base = mo5u
  modified_base 653: mod_base = mo5u
  modified_base 664: mod_base = mo5u
  modified_base 665: mod_base = mo5u
  modified_base 688: mod_base = mo5u
  modified_base 694: mod_base = mo5u
  modified_base 695: mod_base = mo5u
  modified_base 700: mod_base = mo5u
  modified_base 701: mod_base = mo5u
  modified_base 704: mod_base = mo5u
  modified_base 707: mod_base = mo5u
  modified_base 708: mod_base = mo5u
  modified_base 718: mod_base = mo5u
  modified_base 721: mod_base = mo5u
  modified_base 730: mod_base = mo5u
  modified_base 748: mod_base = mo5u
  modified_base 758: mod_base = mo5u
  modified_base 763: mod_base = mo5u
  modified_base 766: mod_base = mo5u
  modified_base 784: mod_base = mo5u
  modified_base 802: mod_base = mo5u
  modified_base 803: mod_base = mo5u
  modified_base 808: mod_base = mo5u
  modified_base 809: mod_base = mo5u
  modified_base 815: mod_base = mo5u
  modified_base 817: mod_base = mo5u
  modified_base 827: mod_base = mo5u
  modified_base 829: mod_base = mo5u
  modified_base 862: mod_base = mo5u
  modified_base 868: mod_base = mo5u
  modified_base 869: mod_base = mo5u
  modified_base 874: mod_base = mo5u
  modified_base 875: mod_base = mo5u
  modified_base 878: mod_base = mo5u
  modified_base 880: mod_base = mo5u
  modified_base 881: mod_base = mo5u
  modified_base 895: mod_base = mo5u
  modified_base 898: mod_base = mo5u
  modified_base 904: mod_base = mo5u
  modified_base 931: mod_base = mo5u
  modified_base 937: mod_base = mo5u
  modified_base 940: mod_base = mo5u
  modified_base 958: mod_base = mo5u
  modified_base 974: mod_base = mo5u
  modified_base 976: mod_base = mo5u
  modified_base 979: mod_base = mo5u
  modified_base 982: mod_base = mo5u
  modified_base 983: mod_base = mo5u
  modified_base 985: mod_base = mo5u
  modified_base 1003: mod_base = mo5u
  modified_base 1010: mod_base = mo5u
  modified_base 1013: mod_base = mo5u
  modified_base 1037: mod_base = mo5u
  modified_base 1039: mod_base = mo5u
  modified_base 1043: mod_base = mo5u
  modified_base 1045: mod_base = mo5u
  modified_base 1046: mod_base = mo5u
  modified_base 1051: mod_base = mo5u
  modified_base 1052: mod_base = mo5u
  modified_base 1057: mod_base = mo5u
  modified_base 1058: mod_base = mo5u
  modified_base 1061: mod_base = mo5u
  modified_base 1072: mod_base = mo5u
  modified_base 1075: mod_base = mo5u
  modified_base 1081: mod_base = mo5u
  modified_base 1112: mod_base = mo5u
  modified_base 1114: mod_base = mo5u
  modified_base 1117: mod_base = mo5u
  modified_base 1129: mod_base = mo5u
  modified_base 1135: mod_base = mo5u
  modified_base 1136: mod_base = mo5u
  modified_base 1153: mod_base = mo5u
  modified_base 1159: mod_base = mo5u
  modified_base 1160: mod_base = mo5u
  modified_base 1162: mod_base = mo5u
  modified_base 1177: mod_base = mo5u
  modified_base 1180: mod_base = mo5u
  modified_base 1184: mod_base = mo5u
  modified_base 1184: mod_base = mo5u
  modified_base 1186: mod_base = mo5u
  modified_base 1187: mod_base = mo5u
  modified_base 1190: mod_base = mo5u
  modified_base 1193: mod_base = mo5u
  modified_base 1207: mod_base = mo5u
  modified_base 1208: mod_base = mo5u
  modified_base 1220: mod_base = mo5u
  modified_base 1226: mod_base = mo5u
  modified_base 1237: mod_base = mo5u
  modified_base 1241: mod_base = mo5u
  modified_base 1253: mod_base = mo5u
  modified_base 1256: mod_base = mo5u
  modified_base 1258: mod_base = mo5u
  modified_base 1265: mod_base = mo5u
  modified_base 1267: mod_base = mo5u
  modified_base 1273: mod_base = mo5u
  modified_base 1274: mod_base = mo5u
  modified_base 1292: mod_base = mo5u
  modified_base 1307: mod_base = mo5u
  modified_base 1316: mod_base = mo5u
  modified_base 1325: mod_base = mo5u
  modified_base 1327: mod_base = mo5u
  modified_base 1400: mod_base = mo5u
  modified_base 1402: mod_base = mo5u
  modified_base 1424: mod_base = mo5u
  modified_base 1468: mod_base = mo5u
  modified_base 1520: mod_base = mo5u
  modified_base 1526: mod_base = mo5u
  modified_base 1528: mod_base = mo5u
  modified_base 1535: mod_base = mo5u
  modified_base 1546: mod_base = mo5u
  modified_base 1553: mod_base = mo5u
  modified_base 1559: mod_base = mo5u
  modified_base 1561: mod_base = mo5u
  modified_base 1562: mod_base = mo5u
  modified_base 1571: mod_base = mo5u
  modified_base 1580: mod_base = mo5u
  modified_base 1592: mod_base = mo5u
  modified_base 1594: mod_base = mo5u
  modified_base 1598: mod_base = mo5u
  modified_base 1600: mod_base = mo5u
  modified_base 1601: mod_base = mo5u
  modified_base 1615: mod_base = mo5u
  modified_base 1618: mod_base = mo5u
  modified_base 1624: mod_base = mo5u
  modified_base 1625: mod_base = mo5u
  modified_base 1642: mod_base = mo5u
  modified_base 1660: mod_base = mo5u
  modified_base 1661: mod_base = mo5u
  modified_base 1664: mod_base = mo5u
  modified_base 1667: mod_base = mo5u
  modified_base 1678: mod_base = mo5u
  modified_base 1688: mod_base = mo5u
  modified_base 1700: mod_base = mo5u
  modified_base 1711: mod_base = mo5u
  modified_base 1712: mod_base = mo5u
  modified_base 1724: mod_base = mo5u
  modified_base 1726: mod_base = mo5u
  modified_base 1736: mod_base = mo5u
  modified_base 1738: mod_base = mo5u
  modified_base 1739: mod_base = mo5u
  modified_base 1741: mod_base = mo5u
  modified_base 1742: mod_base = mo5u
  modified_base 1744: mod_base = mo5u
  modified_base 1745: mod_base = mo5u
  modified_base 1760: mod_base = mo5u
  modified_base 1766: mod_base = mo5u
  modified_base 1768: mod_base = mo5u
  modified_base 1780: mod_base = mo5u
  modified_base 1784: mod_base = mo5u
  modified_base 1787: mod_base = mo5u
  modified_base 1802: mod_base = mo5u
  modified_base 1811: mod_base = mo5u
  modified_base 1817: mod_base = mo5u
  modified_base 1829: mod_base = mo5u
  modified_base 1838: mod_base = mo5u
  modified_base 1850: mod_base = mo5u
  modified_base 1871: mod_base = mo5u
  modified_base 1874: mod_base = mo5u
  modified_base 1879: mod_base = mo5u
  modified_base 1880: mod_base = mo5u
  modified_base 1882: mod_base = mo5u
  modified_base 1895: mod_base = mo5u
  modified_base 1897: mod_base = mo5u
  modified_base 1903: mod_base = mo5u
  modified_base 1904: mod_base = mo5u
  modified_base 1910: mod_base = mo5u
  modified_base 1922: mod_base = mo5u
  modified_base 1925: mod_base = mo5u
  modified_base 1942: mod_base = mo5u
  modified_base 1949: mod_base = mo5u
  modified_base 1958: mod_base = mo5u
  modified_base 1960: mod_base = mo5u
  modified_base 1961: mod_base = mo5u
  modified_base 1970: mod_base = mo5u
  modified_base 1976: mod_base = mo5u
  modified_base 1991: mod_base = mo5u
  modified_base 1993: mod_base = mo5u
  modified_base 1994: mod_base = mo5u
  modified_base 1999: mod_base = mo5u
  modified_base 2014: mod_base = mo5u
  modified_base 2017: mod_base = mo5u
  modified_base 2018: mod_base = mo5u
  modified_base 2020: mod_base = mo5u
  modified_base 2032: mod_base = mo5u
  modified_base 2033: mod_base = mo5u
  modified_base 2035: mod_base = mo5u
  modified_base 2038: mod_base = mo5u
  modified_base 2045: mod_base = mo5u
  modified_base 2050: mod_base = mo5u
  modified_base 2063: mod_base = mo5u
  modified_base 2072: mod_base = mo5u
  modified_base 2081: mod_base = mo5u
  modified_base 2085: mod_base = mo5u
  modified_base 2090: mod_base = mo5u
  modified_base 2095: mod_base = mo5u
  modified_base 2122: mod_base = mo5u
atgtccctgtggcagcccctggtgctggtgctgctggtgctgggctgctgcttcgccgccccaaggcagcgccagagcacactggtgctgttccccggcgatctccggaccaacctgacagacagacagctggccgaggagtacctgtacaggtacggctacacccgcgtggccgagatgcggggcgagtccaagagcctgggcccagccctgctgctgctccagaagcagctgtccctgcccgagacaggcgagctggacagcgccaccctgaaggccatgagaacaccaaggtgcggcgtgcccgacctgggccgcttccagaccttcgagggcgacctgaagtggcaccaccacaacatcacatactggatccagaactactccgaggatctcccacgggccgtgatcgacgacgccttcgccagagccttcgccctgtggagcgccgtgacccccctgacattcaccagggtgtactcccgcgacgccgacatcgtgatccagttcggcgtggccgagcacggcgacggctacccattcgacggcaaggacggcctgctggcccacgccttccccccaggccccggcatccagggcgacgcccacttcgacgacgacgagctgtggagcctgggcaagggcgtggtggtgccaacacggttcggcaacgccgacggcgccgcctgccacttccccttcatcttcgagggcagatcctacagcgcctgcaccacagacgggcggtccgacggcctgccatggtgcagcaccacagccaactacgacaccgacgaccgcttcggcttctgcccctccgagcggctgtacacacaggacggcaacgccgacggcaagccatgccagttccccttcatcttccagggccagagctactccgcctgcaccacagacggcagaagcgacggctacaggtggtgcgccaccacagccaactacgaccgcgacaagctgttcggcttctgcccaacccgggccgactccacagtgatgggcggcaacagcgccggcgagctgtgcgtgttccccttcaccttcctgggcaaggagtactccacatgcaccagcgagggcagaggcgacggcaggctgtggtgcgccacaacctccaacttcgacagcgacaagaagtggggcttctgcccagaccagggctactccctgttcctggtggccgcccacgagttcggccacgccctgggcctggaccacagctccgtgcccgaggccctgatgtacccaatgtaccgcttcacagagggccccccactgcacaaggacgacgtgaacggcatccggcacctgtacggccccagaccagagcccgagccaaggcccccaaccacaaccacaccccagccaaccgcccccccaacagtgtgccccaccggcccacccacagtgcacccaagcgagcgccccaccgccggcccaacaggccccccatccgccggccccaccggcccacccacagccggcccaagcaccgccaccaccgtgcccctgtccccagtggacgacgcctgcaacgtgaacatcttcgacgccatcgccgagatcggcaaccagctgtacctgttcaaggacggcaagtactggcggttcagcgagggcagaggctccaggccccagggcccattcctgatcgccgacaagtggcccgccctgccacgcaagctggacagcgtgttcgaggagcggctgtccaagaagctgttcttcttcagcggcagacaggtgtgggtgtacaccggcgcctccgtgctgggccccaggcgcctggacaagctgggcctgggcgccgacgtggcccaggtgaccggcgccctgcggagcggcagaggcaagatgctgctgttctccggcaggcgcctgtggcggttcgacgtgaaggcccagatggtggaccccagaagcgcctccgaggtggacaggatgttccccggcgtgcccctggacacccacgacgtgttccagtaccgcgagaaggcctacttctgccaggaccggttctactggagagtgagctccaggagcgagctgaaccaggtggaccaggtgggctacgtgacctacgacatcctgcagtgccccgaggactaa

(19) United States
(12) Patent Application Publication
(10) Pub. No.: US 2026/0091058 A1
(43) Pub. Date: Apr. 2, 2026

(54) ENHANCED MACROPHAGES

(71) Applicant: Resolution Therapeutics Limited, London (GB)

(72) Inventors: Lara CAMPANA, London (GB); Kayleigh THIRLWELL, London (GB); Niya ALEKSANDROVA ALEKSIEVA, London (GB)

(73) Assignee: Resolution Therapeutics Limited, London (GB)

(21) Appl. No.: 19/112,928

(22) PCT Filed: Sep. 27, 2023
(86) PCT No.: PCT/EP2023/076692

§ 371 Date: Mar. 18, 2025

(30) Foreign Application Priority Data
Sep. 27, 2022 (GB) 2214084.2
Sep. 27, 2022 (GB) 2214139.4

(51) Int. Cl. A61K 35/15; A61K 38/48; C12N 15/87

(52) U.S. Cl. CPC A61K 35/15; A61K 38/4886; C12N 15/87

(57) ABSTRACT

The present invention relates to a macrophage engineered to express a Matrix Metallopeptidase and therapeutic uses thereof. Such a macrophage may be for use in treatment of an inflammatory condition comprising a fibrotic element in a subject such as inflammatory organ damage. The inflammatory condition may be acute or chronic and may involve a fibrotic element.

### FIELD OF THE INVENTION

The present invention relates to a macrophage, genetically engineered to express Matrix Metallopeptidase 9 (MMP9) and/or Matrix Metallopeptidase 12 (MMP12). The macrophage is preferably engineered with exogenous nucleic acids encoding MMP9 and/or MMP12. Such a macrophage may be for use in treatment of an inflammatory condition comprising a fibrotic element in a subject such as inflammatory organ damage. The inflammatory condition may be acute or chronic and may involve a fibrotic element. The present invention further relates to populations of, compositions comprising, and methods using, such macrophages or populations thereof. The present invention further relates to a method of engineering such macrophages e.g. comprising transient transfection with a MMP9 DNA or mRNA construct. The macrophage may be a human macrophage.

### BACKGROUND TO THE INVENTION

Fibrosis is the final common pathway of chronic disease of various aetiologies, including toxic damage, viral infections, metabolic and genetic diseases, and autoimmune diseases. Acute, self-limiting fibrosis has likely evolved as a reversible and protective response to injury. The balance between self-limited and excessive fibrosis is finely regulated by multiple pathways and systems, and is essentially dependent on the duration and repetition of the injury. A paradigm for the biology of fibrosis generation and remodelling is offered by the liver. End-stage, chronic liver fibrosis also known as cirrhosis, is a life-threatening condition^{1,2}. Mortality due to liver disease is the only leading cause of death that has steadily increased year on year since the 1970s in the United Kingdom and it remains a major health burden worldwide³.

Hepatic decompensation (HD), defined by the acute development of one or more major complications of cirrhosis (i.e., ascites, encephalopathy, gastrointestinal variceal haemorrhage, and spontaneous bacterial peritonitis), represents a morbid advancement during the clinical course of liver cirrhosis [Trebicka 2020]³¹ and is the most common cause of hospitalisation in patients with liver cirrhosis [Moreau 2013]³². Patients with HD are at high risk for short-term death [Moreau 2013]³². The first episode of HD (also referred to herein as a first hepatic decompensation event), which often requires hospitalisation, signals the transition from compensated to decompensated cirrhosis. Decompensated cirrhosis is characterised by recurrent episodes of HD. HD has two distinct clinical presentations, depending on the presence or absence of other organ failures and the grade of systemic inflammation. The presence of multiple organ failures and high-grade systemic inflammation is the hallmark of acute-on-chronic liver failure (ACLF), a syndrome associated with a very high 28-day mortality rate. HD associated with moderate systemic inflammation not involving additional organs has a lower 28-day mortality rate (˜2%, although this increases to 10% at 90-days suggesting a heterogeneity of clinical course in patients with HD) [Trebicka 2020]³¹ but still portends a poor outcome over the ensuing years.

The only therapeutic approaches for liver injury entail removal of the injurious stimuli (e.g. administration of an efficacious anti-viral therapy) and liver transplantation. Treatment of liver failure arising from either acute or chronic injury is limited to supportive care and/or transplantation, the latter of which requires donors. Currently there is a deficit of available organ donors and the surgical procedure carries significant morbidity and mortality risks. In addition, patients are also committed to life-long immunosuppression. Furthermore, despite the relative success of therapeutic interventions for specific aetiologies (e.g., novel antiviral therapy for hepatitis C virus infection, alcohol abstinence for alcoholic liver disease), many diseases (e.g. NASH) have not got approved medical therapies and patients often present to medical attention late when cirrhosis and related complications have already occurred [Starkey 2019]³³. Thus, there are no specific therapies to treat hepatic cirrhosis and so delivering an effective anti-fibrotic therapy is therefore a major unmet clinical need for both chronic and acute liver damage⁴⁻⁶.

Macrophages (Mφ) play a pivotal role in the inflammatory response in the injured liver. In the liver there are two main populations of Mφ: (i) resident macrophages (Kupffer Cells, KCs), and (ii) infiltrating macrophages. KCs carry out patrolling functions in the liver sinusoids to phagocytose microbial debris that reaches the liver via the sinusoidal capillaries in homeostatic conditions. During the early phases of liver damage, KCs express chemokines such as CCL2 and CCL5, thereby contributing to the recruitment of monocytes from the circulation^{2,7}. The number of KCs decreases during fibrosis; they then repopulate the liver in the recovery phase of self-limiting fibrosis⁸. Infiltrating monocyte derived Mφ (MDMs) play a major role in the response to liver damage. Infiltrating Mφ are recruited via the CCR2/CCL2 axis; once in the liver parenchyma, they locate along the fibrotic septa in the early stages of liver fibrosis and may promote fibrosis by releasing factors such as TGF-β, IL1, PDGF and CCL2 that activate hepatic stellate cells and worsen inflammation. This may suggest a detrimental role for Mφ in progressive fibrosis. However, if Mφ are depleted at the onset of fibrosis remodelling, the remodelling process fails, and the liver fibrosis persists. It is now widely accepted that macrophages play a dual role in the establishment and resolution of fibrosis^{2,8-11}.

Due to the suggested role of macrophages in healing of fibrosis, it has been considered that a macrophage cell therapy could be beneficial for the reduction of chronic liver fibrosis. It has been shown that mouse bone marrow-derived macrophages (BMDMs) improve liver fibrosis when injected into a mouse model of chronic liver fibrosis¹². Similar results have been replicated using human monocyte-derived macrophages (hMDMs) in immunodeficient mouse models of chronic liver fibrosis¹³. Furthermore, a GMP-graded cell culture protocol¹⁴ is currently used to generate hMDMs for autologous transplantation into cirrhotic patients in an ongoing phase II trial (MATCH, Macrophage therapy for liver disease, ISRCTN10368050).

The hMDMs currently utilised in the clinic are not enhanced for anti-fibrotic function¹⁴, which may limit the extent and duration of their therapeutic effect. An ideal therapeutic macrophage to be used in inflammatory organ damage with a fibrotic component should have anti-fibrotic properties.

Matrix metalloproteinases (MMPs), also known as matrix metalloproteases or matrixins, are a family of zinc-dependent polypeptidases that collectively degrade various proteins in the extracellular matrix (ECM). MMPs have a magnitude of roles in vivo, making them extremely complex to understand. In the context of fibrosis, it is reported that MMPs are contributory, however, they are also pivotal in fibrosis regression through their matrix remodelling capacity. Their dual role in fibrosis progression and regression can somewhat be appreciated through their complex in vivo activity ranging from cytokine and chemokine activation, immune cell recruitment and activation to extracellular matrix degradation¹⁵. In addition, the activation and subsequent activity of MMPs is highly regulated, largely by tissue inhibitors of metalloproteinases (TIMPS), adding to the difficulty of understanding their activity in fibrosis. Experimental models of lung fibrosis have exhibited an increase in MMP9 (a type IV collagenase targeting collagen) activity which has also been associated with the disruption of the alveolar epithelial membrane, indicating a putative pro-fibrotic role of MMP9 in lung injury. However, in a bleomycin-induced model of lung fibrosis, MMP9-null mice develop similar lung fibrosis to wild-type littermates, although the lungs of the MMP9 deficient mice showed limited alveolar bronchiolisation. Overall, the precise role of MMPs in fibrosis is not completely understood. Moreover, in vitro derived hMDMs express negligible levels of MMPs; plus, hardly any activity is detected in the conditioned media of these cells.

Little has been reported thus far in the field of MMP genetic engineering. MMP9 transfection in THP1 cells has been utilised as a research tool to understand the inflammatory responses of macrophages in atherosclerosis¹⁶. Therapeutically, the overexpression of MMP9 in iPSC via lentiviral transduction has been utilised to provide enhanced repair to damaged myocardium¹⁷. MMP12 (an elastase targeting soluble and insoluble elastin) has been overexpressed in endothelial progenitor cells for use in a melanoma cell therapy¹⁸. None of the previous work performed with MMP transfection of human macrophages, as far as the present inventors are aware, has resulted in macrophages with anti-fibrotic properties. Cabrera et al. (2007) demonstrated overexpression of MMP9 in macrophages in transgenic mice challenged with bleomycin resulted in attenuation of fibrosis²¹. Differences in the levels of TIMP-1 between the transgenic mice and wild-type mice were also observed. However, a difference in expression levels or activity of other matrix metalloproteinases was not reported.

Introduction to the Invention Typically, human macrophages derived in vitro from monocytes (hMDM) will express MMPs under certain physiological conditions. As shown in FIGS. 1 and 4, in vitro derived hMDMs express negligible levels of MMPs; plus, hardly any activity is detected in the conditioned media of these cells (FIG. 2). Herein, the inventors enhance macrophage anti-fibrotic properties thanks to the enhanced expression of MMPs in general by means of transient transfection with MMP9 or MMP12 coding sequences, as a proof of principle of how the additional expression of these genes delivers a promising product to help fibrotic conditions such as acute and chronic organ damage resolution, delivering macrophages that secrete active MMPs. Some MMPs have a role in helping fibrosis remodelling. However, the effects of the additional expression MMP9 or MMP12 on the general MMP expression in a macrophage is currently unknown.

The inventors show that the most surprising feature of our MMP-transfected macrophages is the ability to co-upregulate other MMPs beyond the MMP of choice: in FIG. 4B the inventors show that MMP9 transfected hMDMs strongly upregulate several other MMPs, such as MMP1, 8 and 10, which could contribute significantly to the anti-fibrotic effect of such macrophages as cell therapy. For example, MMP8 is normally contained in neutrophils granules and digest collagen I, II, and III, thereby being of particular interest for liver fibrosis²⁷, where most of the collagen in the fibrotic septa is collagen I and III²⁸.

The inventors, therefore, also provide a macrophage engineered with a sequence encoding a specific MMP, MMP9 and/or MMP12. Interestingly the overall matrix metalloproteases activity of MMP9 and MMP12 transfected hMDMs increases significantly, this effect is not simply limited to the proteinase transfected.

As demonstrated herein, the MMP-transfected macrophages maintain cell surface marker expression of macrophage-specific markers such as CD14, CD206 and 25F9 (FIG. 3), thereby demonstrating that transfection with MMP9 or MMP12 does not alter their cell identity. However, interestingly, MMP9 transfected cells are much better phagocytes as compared to MMP12 transfected cells (FIG. 5). This further demonstrates the versatility of the therapeutic approach described herein, as the appropriate MMP with the appropriate combination of anti-fibrotic and pro-phagocytic capacity can be chosen depending on the disease target.

Taken together, these data support the use of MMP9 and/or MMP12 Trx hMDMs as a therapeutic product in the treatment and/or prevention of fibrotic conditions (e.g. related to inflammatory organ damage). The MMP9 or MMP12 expression (e.g., via transfection) is envisaged to reinforce the anti-fibrotic (via matrix metalloprotease activity increase) function of macrophage-based therapeutic products.

Chronic inflammatory organ damage is often associated with fibrosis, such as for example in chronic liver disease. Therefore, a pro-restorative therapeutic macrophage with anti-inflammatory and anti-fibrotic functions would be beneficial.

Macrophages engineered to additionally express MMP9 and/or MMP12, for example by engineering with a nucleic acid encoding MMP9 or MMP12, exhibit an enhancement in overall MMP activity, not just MMP9/MMP12 activity.

Ramachandran et al. (2012)⁸ and Ramachandran et al., Gut. 2011 April; 60; A56 assessed macrophages in mice following liver injury and identified a subset of macrophages associated with the resolution of fibrosis. The cells associated with resolution of fibrosis had increased expression of various genes associated with clearance of cellular debris and anti-fibrotic pathway, and reduced expression of pro-fibrotic and pro-inflammatory genes. Among the genes whose expression increased were MMP9 and MMP12. Moore J K, et al., (2015)¹³ demonstrated that monocytes from cirrhotic patients may be differentiated into macrophages in vitro and that said macrophages may express a suite of genes associated with anti-fibrotic pathways, such as MMP9 and 12. Li Y H, et al., Cell Death Discov. 2021 Sep. 13; 7(1):239 also identifies upregulation of MMPs, such as MMP9 and 12, is a key mechanism by which anti-fibrotic macrophages resolve fibrosis, but this was in addition to the change in expression of other genes, such as the downregulation of pro-inflammatory and fibrogenic cytokines. Ricardo Lamy et al., Invest. Ophthalmol. Vis. Sci. 2018; 59(9):4338 identifies MMP12 as having anti-fibrotic properties, and that knockout of this MMP results in increased fibrosis. However, it also identifies that MMP12 regulates the expression of other genes such as CCL2, and therefore that affecting the expression of MMP12 alone could nonetheless have unpredictable consequences given the complex interactions regulating gene expression. Wang M et al., Theranostics. 2020 Jan. 1; 10(1):36-49. indicates that MMP12 may exert anti-fibrotic effects by suppressing hepatic stellate cell activation and pro-inflammatory cytokine release.

However, none of these documents relate to engineered cells. Indeed, none of these documents exemplify or even suggest macrophages engineered to express MMP9 and/or MMP12, or that cells engineered to express these genes alone (in the absence of the other identified anti-fibrotic genes) would be therapeutically beneficial, given the complex nature of the changes in gene expression and multiple contributors to fibrosis in vivo. It is also known that overexpression of MMPs may cause cells to upregulate Tissue Inhibitors of Metalloproteinases (TIMPs), therefore it would have been expected that engineering a cell with MMPs would cause TIMP upregulation and negate any advantageous technical effect resulting from expression of MMPs.

WO2019/118888A1 (TREATMENT OF FIBROSIS WITH GENETICALLY-ENGINEERED MACROPHAGES) describes macrophages engineered for treating fibrosis. For example, a genetically-engineered macrophage, comprising: a recombinant extracellular matrix (ECM) targeting protein; and/or a recombinant protease. The recombinant protease may be a matrix metalloproteinase (MMP), e.g. one from a long list of possible MMPs. However, neither MMP9 nor MMP12 are exemplified therein, and therefore the effect on the activity levels of the MMPs as a whole is not observed. Moreover, the engineered cells described therein are tested only in models of lung or cardiac fibrosis.

WO2022/047119A1 (MODIFIED IMMUNE CELLS FOR FIBROSIS AND INFLAMMATION) describes a modified immune cell comprising one or more nucleic acid sequences encoding: (i) at least one exogenous fibrolytic agent, and/or (ii) at least one exogenous anti-inflammatory agent. The at least one exogenous fibrolytic agent may comprise a matrix metallopeptidase (MMP), e.g. one or more from a long list of possible MMPs including MMP9 and MMP12. The at least one exogenous anti-inflammatory agent may comprise e.g. a cytokine, a chemokine, or a pentraxin, and the cytokine may comprise e.g. IL-10, IL-4, IL-13, and/or TGF-beta. The modified immune cell may comprise a macrophage. However, again neither MMP9 nor MMP12 are exemplified therein, and therefore the effect on the activity levels of the MMPs as a whole is not observed.

The present inventors have demonstrated for the first time the unexpected advantageous effects of macrophages engineered to express MMP9 or MMP12, for example in the treatment of fibrotic and/or inflammatory diseases/conditions.

### SUMMARY OF THE INVENTION

The present invention relates to a genetically engineered macrophage with enhanced anti-fibrotic functions by means of transfection with exogenous sequences encoding MMP9 and/or MMP12. Such macrophages are suitable for use in treating an inflammatory condition, preferably an inflammatory condition with a fibrotic element. The genetically engineered macrophage described herein displays multiple favourable characteristics, including: (1) proficient secretion of active MMP9 and/or MMP12; (2) upregulation of functional MMP9 and/or MMP12 without the upregulation of TIMPs; and most surprisingly, (3) co-upregulation of other MMPs beyond the transfected MMP. This surprising effect demonstrates the versatility of the present invention, as the appropriate exogenous MMP (MMP9 or MMP12) with the appropriate combination of anti-fibrotic and pro-phagocytic capacity can be selected depending on the disease target. The invention further relates to a population of genetically engineered macrophages, a method of generating the macrophage(s), and compositions for use thereof.

The macrophage of the invention may suitably be a human macrophage.

According to a first aspect of the present invention, there is provided an engineered macrophage, wherein the macrophage is engineered to express MMP9 or MMP12, preferably by the provision of additional or exogenous sequences encoding these proteins. Such engineered macrophages may have utility in therapy, for example, for use in treating an inflammatory condition and/or a fibrotic condition in a subject.

In one embodiment, the present invention provides an engineered macrophage comprising one or more exogenous coding sequences for MMP9 and/or MMP12. Said exogenous coding sequence may be any suitable nucleic acid sequence. Said exogenous coding sequence may be present in the cytoplasm or nucleus as an extrachromosomal nucleic acid or integrated into the macrophage genome.

In some embodiments, endogenous MMP9 and/or MMP12 gene expression can be stimulated by genetic engineering. For example, gene editing techniques such as CRISPR can be used to turn on and off the endogenous genes that encode these MMPs, generating an engineered macrophage that expresses MMP12 and/or MMP9 under conditions in which it would not otherwise express these proteins. This may be done by altering the promoter sequence, for example.

In some embodiments, the macrophage is engineered by introducing an exogenous nucleic acid encoding MMP9. In some embodiments, the macrophage is engineered through gene editing, optionally wherein the gene editing uses a CRISPR-Cas9 system, or genetic modification which increases the expression of MMP9. In some particular embodiments, the engineering may comprise editing of a promoter sequence to increase the expression of MMP9, preferably wherein the promoter of MMP9 is edited. In other particular embodiments, the engineering comprises gene editing or genetic modification which increases the expression of a transcription factor which upregulates the expression of MMP9, optionally wherein the transcription factor upregulates the expression of MMP9 by interacting with or binding the promoter of MMP9. In other particular embodiments, the gene editing or genetic modification causes the downregulation of, or inhibits the activity of, an miRNA which represses the expression of MMP9, optionally wherein the gene editing or genetic modification comprises deleting sequences encoding the miRNA.

Natural non-engineered macrophages are capable of expressing MMP9 and/or MMP12 under relevant physiological conditions. The present invention does not relate to these natural non-engineered macrophages, but instead to engineered macrophages wherein the activity levels of MMPs are raised, elevated or enhanced in comparison to natural cells. Accordingly, in one aspect of the invention, there is provided an engineered macrophage, wherein the macrophage is engineered to express MMP9 at a higher level than is expressed endogenously in non-engineered macrophages.

In other embodiments, the macrophage may be engineered to turn on endogenous genes that encode MMP9 and/or MMP12. In any case, the macrophages of the disclosure have been modified through alterations in expression levels by any means and thus are referred to as engineered macrophages.

As described, the engineered macrophage is provided with an additional or exogenous MMP9 or MMP12 coding sequence to enhance the overall activity of the MMPs, suitably the activity is increased between 1.2 and 1.5 times wild type, such as 1.2, 1.3, 1.4 or 1.5 times the natural level/wild type.

The macrophage is engineered to express MMP9 or MMP12. This expression may be driven from the endogenous genes in some embodiments. In other embodiments, the macrophage is engineered to contain exogenous coding sequence(s) for MMP9 or MMP12. It may be preferred that the macrophage over-expresses MMP9. It may be preferred that the macrophage over-expresses MMP12. Expression levels of MMP9 and/or MMP12 are increased when compared to a non-transfected macrophage.

In some embodiments, the macrophage that is engineered is a human monocyte-derived macrophage. In some embodiments, the macrophage is derived from monocytes by culturing in the presence of MCSF. Suitable culturing conditions are discussed further below

In some embodiments, the baseline macrophages (i.e., pre-engineered or natural) are referred to as unpolarised human monocyte-derived macrophages, also terms “resting” macrophages.

In some embodiments, the macrophage that is engineered is a macrophage derived from an iPSC. There are various methods known in the art to derive macrophages from iPSC. At a baseline, these would also be unpolarised or resting.

Said macrophage is isolated, thus said cell may be described as ex vivo.

Suitably, said macrophage is suitable for use in treating an inflammatory condition, preferably an inflammatory condition with a fibrotic element.

As used in the Examples, expression levels of the proteins were quantified at between 16 to 24 hours post-transfection. Expression levels as recited herein are given for a population of macrophages at a concentration of 4×10⁶/ml (which equates to 2×10⁶ cells per cm²). In the Examples, the macrophages were transfected, isolated by centrifugation, re-suspended in TexMACs buffer supplemented with IL-3 and IL-14, and incubated at 37° C. under 5% CO₂. Those skilled in the art would be aware of equivalent conditions suitable to determine secreted protein concentration.

In some embodiments, the macrophage is engineered to express MMP9, wherein the secreted MMP9 protein level is greater than about 200 ng/ml. Suitably, the secreted MMP9 protein is greater than about: 300 ng/ml or 400 ng/ml or 500 ng/ml or 600 ng/ml or 700 ng/ml or 800 ng/ml or 900 ng/ml or 1,000 ng/ml. Suitably the secreted MMP9 protein level is between about 200 ng/ml and 2000 ng/ml. Suitably the engineered macrophage has a secreted MMP9 protein level that is greater than the average level of secreted MMP9 protein in non-engineered macrophages. Suitably, the overall MMP activity of the engineered macrophage of the invention is also higher than the overall MMP activity of a non-engineered macrophage engineered. Suitably, these protein levels may be measured by culturing the macrophages as described above, wherein the concentration of macrophages in the medium is 4×10⁶ cells/ml, which equates to 2×10⁶ cells/cm², and measuring the concentration of the protein in the culture medium. Accordingly, in some embodiments, the secreted MMP9 protein level is greater than 200 ng/ml when cultured in vitro at a cell concentration of 4×10⁶/ml.

In some embodiments, the macrophage is engineered to express MMP12, wherein the secreted MMP12 protein level is greater than about 200 ng/ml. Suitably, the secreted MMP12 protein is greater than about: 300 ng/ml or 400 ng/ml or 500 ng/ml or 600 ng/ml or 700 ng/ml or 800 ng/ml or 900 ng/ml or 1,000 ng/ml. Suitably the secreted MMP12 protein level is between about 200 ng/ml and 2000 ng/ml. Suitably the engineered macrophage has a secreted MMP12 protein level that is greater than the average level of secreted MMP12 protein in non-engineered macrophages. Suitably, the overall MMP activity of the engineered macrophage of the invention is also higher than the overall MMP activity of a non-engineered macrophage. Suitably, these MMP12 protein levels may be measured by culturing the macrophages as described above, wherein the concentration of macrophages in the medium is 4×10⁶ cells/ml, which equates to 2×10⁶ cells/cm², and measuring the concentration of the protein in the culture medium.

Known viral vectors for transfecting macrophages include lentivirus, adenoviruses and adeno-associated viruses (AAV).

As used herein, “coding” refers to the ability of sequences of nucleotides, such as a gene, a cDNA, or an mRNA, to serve as templates for synthesis of macromolecules in biological processes such as a defined sequence of amino acids. Thus a coding sequence may be any suitable nucleic acid sequence which provides the instructions to synthesise the relevant entity (e.g., MMP9 or MMP12).

As used herein, “exogenous” refers to any material, notably genetic material, introduced from or produced outside a particular cell. As used herein, in some embodiments, the exogenous coding sequence or engineered macrophage encode/express MMP9 or MMP12. In some embodiments, the MMP9 or MMP12 are human. It will be understood by those skilled in the art that variations to the sequence of these genes/coding sequences is also encompassed in this invention. Ideally, the genes/coding sequences are human. The genes/coding sequences may be codon optimised. The gene/coding sequences may be adjusted, and it will be appreciated if the reference sequence is RNA, then a different nucleotide may be present in a DNA vector.

Preferably, the sequence of the MMP9 coding sequence provided is at least 80% similar or homologous to SEQ ID NO. 1 (Homo sapiens matrix metallopeptidase 9 (MMP9), mRNA), preferably at least 85%, at least 90%, at least 95%, 96%, 97%, 98%, 99% similar to SEQ ID NO. 1.

Preferably, the MMP9 protein expressed is preferably at least 80% similar or homologous to the sequence presented as SEQ ID. No. 2, preferably at least 85%, at least 90%, at least 95%, 96%, 97%, 98%, 99% similar to SEQ ID No. 2.

Preferably, the sequence of the MMP12 coding sequence provided is at least 80% similar or homologous to SEQ ID No. 3 (Homo sapiens matrix metallopeptidase 12 (MMP12), mRNA), preferably at least 85%, at least 90%, at least 95%, 96%, 97%, 98%, 99% similar to the SEQ ID No. 3.

Preferably, the MMP12 protein expressed is preferably at least 80% similar or homologous to the sequence presented as SEQ ID. No. 4, preferably at least 85%, at least 90%, at least 95%, 96%, 97%, 98%, 99% similar to SEQ ID No. 4.

In preferred embodiments of the aspects set out in the preceding paragraphs, the coding sequences and proteins are at least 80%, at least 85%, at least 90%, at least 95%, at least 96%, at least 97%, at least 98%, or at least 99% identical to SEQ ID No. 1, 2, 3 or 4, or comprise or consist of SEQ ID No: 1, 2, 3 or 4.

Preferably, the sequence of the MMP9 coding sequence provided is at least 80% similar or homologous to SEQ ID No. 5, preferably at least 85%, at least 90%, at least 95%, 96%, 97%, 98%, 99% similar to SEQ ID No. 5. In a preferred embodiment, the MMP9 coding sequence provided is at least 80%, at least 85%, at least 90%, at least 95%, at least 96%, at least 97%, at least 98%, or at least 99% identical to SEQ ID No. 5, or comprises or consists of SEQ ID No: 5. Preferably, the sequence further comprises a polyA tail, optionally between 65 and 250 residues long, preferably 90 to 120 residues long, preferably about 90 residues long, and/or a 5′ cap.

In some embodiments, the macrophage is non-virally engineered, and is engineered with a nucleic acid vector. In some embodiments, the macrophage is transfected with a DNA vector e.g. a naked DNA vector or plasmid. In some embodiments, the nucleic acid vector is not derived from a viral genome. In some embodiments the macrophage is transfected with one or more free nucleic acids or vectors. In some embodiments, the macrophage is transfected with a RNA vector. In some embodiments, the macrophage is transfected with mRNA.

In some embodiments, the MMP9 or MMP12 is provided to the macrophage as mRNA. Thus, the engineered cell is provided with exogenous mRNA molecules.

In some embodiments, the mRNA molecule(s) contain a poly A tail. A poly-A tail is a long chain of adenine nucleotides that is added to mRNA molecule during RNA processing to increase the stability of the molecule. A suitable poly-A tail is between 65 and 250 residues long, preferably 90 to 120 residues long. The poly-A tail makes the RNA molecule more stable and prevents its degradation.

In some embodiments, the mRNA molecules contains at least one cap. The 5′-cap is a hallmark of eukaryotic mRNA. Chemically, the 5′-cap consists of an inverted 7-methylguanosine connected to the rest of the eukaryotic mRNA via a 5′-5′ triphosphate bridge. This so-called “cap0” contributes to, inter alia, stabilization of eukaryotic mRNA, initiation of translation and mRNA decay. Artificial cap structures have been designed, to increase the success of in vitro translation. Synthetic anti-reverse cap analogues include extra methylation and trinucleotide CleanCap®AG, methylated at first adenosine. At least one cap may be a synthetic cap analogue, preferably a CleanCap®.

In some embodiments, the mRNA is modified RNA. In some embodiments, the mRNA modification comprises a chemical modification of uridine and/or a chemical modification of cytidine. In some embodiments, the mRNA modification comprises pseudouridine, N1-Methylpseudouridine, 5-methoxy-uridine, 5-methyl-cytidine, preferably 5-methoxy-uridine.

In an embodiment, the mRNA is modified with 5-methoxy-uridine and includes at least one CleanCap®.

In some embodiments, the macrophage is engineered using a nucleic acid vector. In some embodiments the macrophage is transfected via electroporation. Other suitable methods of transfection include nucleofection.

In some embodiments, the nucleic acid vector is delivered to the macrophage via nanoparticles. In some embodiments, the nanoparticles are lipid nanoparticles, which may be as described in, for example, U.S. Pat. Nos. 8,058,069, 8,492,359, 8,822,668, 9,364,435, 9,504,651, and 11,141,378.

In some embodiments, the macrophage is autologous or allogenic to the subject. In some embodiments, the macrophage is derived from a progenitor cell, such as an iPSC, a hematopoietic stem cell, or a monocyte.

In some embodiments, the macrophage is derived from monocytes by culturing the latter under appropriate conditions. Such conditions may suitably include:

An in vitro method of producing macrophages comprising:

(b) Culturing monocytes in medium for 3-5 days to produce macrophages, wherein the medium comprises one or more growth factors to stimulate macrophage production;

Wherein step (a) takes place entirely in the same medium.

Suitably the medium is suitable for generating macrophages from monocytes. Suitably the medium is a T-cell medium. Suitably the medium may be selected from: X-Vivo 10, X-Vivo 15, TexMACS, AIMv, RPMI, DMEM, and DMEM/F12. Suitably the medium is TexMACS (Miltenyi).

Suitably the medium is serum-free. Suitably the medium is xenoprotein-free. Suitably the medium is GMP-compliant.

Suitably the medium may contain one or more factors. Suitable factors include growth factors, polysaccharides, cytokines and chemokines. Suitable factors may include: MCSF, GM-CSF. Suitably therefore the factors are growth factors. Suitably, the one or more factors are GMP-compliant. In one embodiment, the medium may comprise one or more growth factors may include MCSF or GM-CSF. Monocytes are most commonly cultured with either MCSF or GM-CSF. Culturing monocytes with GM-CSF skews them towards an “inflammatory” phenotype whereas culturing monocytes with MCSF skews them towards a “pro-restorative” phenotype. In other embodiments, the one or more growth factors does not include the combination of MCSF and GM-CSF. Thus, if M-CSF is used as the growth factor to generate macrophages in any of the methods of the invention, it may be preferred that GM-CSF is not also used to generate the macrophages from monocytes. This applies to the step of culturing monocytes until macrophages are generated.

According to a second aspect of the present invention, there is provided an engineered macrophage wherein the macrophage is engineered to express MMP9, preferably by the provision of additional or exogenous sequences encoding MMP9 for use in treating an inflammatory condition with a fibrotic element in the lung or liver.

According to a third aspect of the present invention, there is provided an engineered macrophage wherein the macrophage is engineered to express MMP12, preferably by the provision of additional or exogenous sequences encoding MMP9 for use in treating an inflammatory condition with a fibrotic element in the liver.

It may be preferred that according to any aspect of the present invention, the inflammatory condition with a fibrotic element is liver cirrhosis.

According to any aspect of the present invention, the use may comprise delivering the engineered macrophage to a subject by systemic administration, suitably by systemic injection. In some embodiments, administration is by local injection, e.g. for kidney and lung. In some embodiments, administration is by nebulizer into the lung. Peripheral vein injection is favoured for liver conditions in order to avoid invasive procedures in cirrhotic patients, for example. Local injection, such as renal artery for kidney conditions may be better tolerated.

It is preferred that the macrophage for use in therapy is engineered ex vivo and delivered to the patient.

However, in some embodiments, the macrophage may be engineered in vivo. In some embodiments, the macrophage is engineered in vivo via the administration to a subject of a preparation of exogenous coding sequence(s) for MMP9 or MMP12 suitable for transfecting a macrophage. The preparation may include any of the exogenous coding sequences discussed herein. Suitable delivery vehicles for in vivo engineering may include targeting molecules for macrophages by virtue of their cell surface markers. Should an in vivo transfection of macrophages be envisaged, localized application of the preparation may be more effective, such as localized injection of the preparation to liver or kidneys, or nebulization to the lungs. Such treatment may be prepared as nanoparticles to assist macrophage targeting.

According to a fourth aspect of the present invention, there is provided a population comprising the engineered macrophages for use according to any aspect of the invention.

According to a fifth aspect of the present invention, there is provided a composition comprising an engineered macrophage for use according to the first, second, or third aspect of the invention or a population of engineered macrophages for use according to the fourth aspect of the invention.

According to a sixth aspect of the present invention, there is provided a method of improving the resolution of fibrosis in a chronic condition, comprising the use of a engineered macrophage according to the first, second, or third aspect of the invention, a population of engineered macrophages according to the fourth aspect of the invention, or a composition according to the fifth aspect of the invention. Alternatively worded, this provides a method of treating such conditions.

According to any aspect of the invention, the engineered macrophage is an ex vivo macrophage, optionally a human ex vivo macrophage.

The engineered macrophage may be derived or isolated from any appropriate source. The engineered macrophage may be isolated from a human. The engineered macrophage may be derived from any suitable precursor cell, including haematopoietic cells. The engineered macrophage may be derived from a human monocyte. It is preferred that the engineered macrophage is derived from a human monocyte. The use of human monocyte-derived macrophages (hMDMs) may therefore be particularly preferred.

Suitably, the matrix metalloproteinase may be selected from MMP9 or MMP12. It is preferred that the matrix metalloproteinase is one or more of MMP9 and MMP12.

According to all aspects of the present invention, the macrophage is engineered with an exogenous nucleic acid comprising a coding sequence encoding an MMP—either MMP9 or MMP12. The coding sequence may be a gene encoding MMP9 or MMP12. The macrophage is therefore capable of expressing said MMP after transfection. In the case of a DNA vector, said coding sequence is preferably operably linked to a promoter to enable expression of said MMP. It may be preferred that said promoter is constitutive for certain applications, or inducible for others, such that the expression of the MMP can be controlled.

Said exogenous nucleic acid may be any suitable nucleic acid. Said exogenous nucleic acid may be present in the cytoplasm or nucleus as an extrachromosomal nucleic acid, or integrated into the macrophage genome. Said exogenous nucleic acid comprising a coding sequence may encode only MMP9 or MMP12, or it may encode both MMP9 and MMP12, or it may encode additional matrix metalloproteinases, or it may encode coding sequences for non-MMP proteins. Where more than one coding sequence is encoded, these may be on the same exogenous nucleic acids, or on different exogenous nucleic acids.

Natural, non-engineered macrophages are capable of expressing MMP9 and/or MMP12 under relevant physiological conditions. However, the present invention does not relate to these natural non-engineered macrophages, but instead to macrophages wherein the expression level of MMP9 or MMP12 has been raised above physiological levels, thereby improving the anti-fibrotic properties of the therapeutic macrophages. As used herein, the inventors describe this as “overexpression” of MMP9 or MMP12. In order to overexpress the matrix metalloproteinases, the macrophage may be engineered such that it possesses additional or exogenous coding sequence(s) for MMP9 and/or MMP12.

The macrophage may be transfected or engineered with a DNA molecule, an RNA molecule, or a non-viral vector. Suitably, the engineered macrophage may be transfected with a DNA vector. Suitably the vector may not be derived from a viral genome. Suitably, the macrophage may be transfected with an mRNA vector. It may be preferred that the macrophage is engineered without viral transfection, since this can polarise or activate the macrophage.

The macrophage may be transfected by any appropriate means. The engineered macrophage may be transfected via electroporation. Alternatively, the macrophage may be transfected by nucleofection. Any suitable means of transfection may be employed.

The macrophage may suitably be engineered by altering the genome of the macrophage by gene editing. Gene editing permits permanent insertion of the exogenous coding sequence. Numerous techniques of gene editing are known, including those that require the use of nucleases. Various nucleases are known that can be exploited to modify the genome, from base editing techniques, prime editing techniques to gene editing. Many nucleases are known—such as Zinc fingers, TALENs and guided nucleases. These may be guided by RNA (“RNA” guided nucleases)—such as the enzymes involved in CRISPR, including but not limited to Cas9, Cas12a, Cas13, Mad7 and the like. The macrophage itself may be subject to gene editing, or a progenitor cell may be gene edited prior to conversion to a macrophage.

As used herein, “coding” refers to the ability of sequences of nucleotides, such as a gene, a cDNA, or an mRNA, to serve as templates for synthesis of macromolecules in biological processes such as a defined sequence of amino acids. Thus a coding sequence may be any suitable nucleic acid sequence which provides the instructions to synthesise the relevant entity (herein MMP9 and/or MMP12). Where more than one coding sequence is introduced, the coding sequences may be included in the same vector/construct or on different vectors/constructs.

As used herein, “exogenous” refers to any material, notably genetic material, introduced from or produced outside a particular cell.

As used herein, the exogenous nucleic acid encodes MMP9 or MMP12, and the engineered macrophage expresses the coding sequence of MMP9 or MMP12 from that exogenous nucleic acid. It will be understood by those skilled in the art that variations to the sequence of these genes/coding sequences is also encompassed in this invention. Ideally, the genes/coding sequences are human. The genes/coding sequences may be codon optimised. The gene/coding sequences may be adjusted.

Preferably, the coding sequence of MMP9 is at least 80% similar to NM_004994.3, preferably at least 85%, at least 90%, at least 95%, 96%, 97%, 98%, 99% similar to NM_004994.3. Preferably, the sequence of MMP12 is at least 80% similar to NM_002426.6, preferably at least 85%, at least 90%, at least 95%, 96%, 97%, 98%, 99% similar to NM_002426.6.

In some embodiments, the macrophage is non-virally engineered, and is contacted with a nucleic acid vector. In some embodiments, the macrophage is transfected with a DNA vector e.g. a naked DNA vector. Suitably, the DNA vector may encode MMP9 or MMP12. In some embodiments, the nucleic acid vector is not derived from a viral genome. In some embodiments the macrophage is transfected with one or more free nucleic acids or vectors. In some embodiments, the macrophage is transfected with an RNA vector. In some embodiments, the macrophage is transfected with mRNA. Suitably, the macrophage is transfected with a single mRNA construct expressing MMP9 or MMP12.

In some embodiments, the MMP9 and/or MMP12 are provided to the macrophage as mRNA. Thus, the engineered cell is provided with exogenous mRNA molecules.

In some embodiments, the mRNA is delivered to the macrophage via nanoparticles. In some embodiments, the nanoparticles are lipid nanoparticles, which may be as described in, for example, U.S. Pat. Nos. 8,058,069, 8,492,359, 8,822,668, 9,364,435, 9,504,651, and 11,141,378.

The engineered macrophage of the present invention may have a repressed STING pathway either temporarily or permanently. Repression of the STING pathway may be achieved by any appropriate means. The STING pathway of the engineered macrophage of the present invention may be repressed by directly inhibiting one or more components of the STING pathway. The engineered macrophage may be contacted with one or more STING pathway inhibitors. The engineered macrophage may be contacted with one or more STING pathway inhibitors after transfection. Alternatively, the engineered macrophage may be contacted with one or more STING pathway inhibitors before or during transfection. The engineered macrophage may be contacted with one or more of the STING pathway inhibitors IL-10, IL-4 and IL-13.

It may be preferred that the engineered macrophage may exhibit a pro-restorative/pro-regenerative phenotype. Such a phenotype is defined further herein. The engineered macrophage may exhibit an M2 or M2-like phenotype and may be anti-inflammatory and anti-fibrotic. The M2 or M2-like phenotype is pro-restorative or pro-regenerative. In some embodiments, a pro-restorative phenotype may be described using one or more of the following markers: an in CD206 and/or CD163, a decrease in inflammatory markers such as CD86 and/or MHC class II (HLA-DR). These increases/decreases are as compared to non-polarised (resting) or pro-inflammatory macrophages. In terms of secretion profile, these macrophages are expected not to express TNFa, IFNg and IL1b, normally associated with a pro-inflammatory and pro-fibrotic profile.

In some embodiments the method comprises contacting the macrophage with an anti-inflammatory treatment after transfection, e.g. IL-4 and IL-13. In some embodiments the method comprises contacting the macrophage with IL-4 and IL-13. In some embodiments, the method comprises electroporation. In some embodiments, the macrophage is contacted with an anti-inflammatory treatment, e.g. IL-4 and IL-13, after electroporation.

The engineered macrophage may be autologous or allogenic to the subject. In some embodiments, the engineered macrophage may be isolated from a human, the human being either the subject or another human. In some embodiments, the macrophage is derived from a progenitor cell, such as a hematopoietic stem cell, or a monocyte. In some embodiments, the macrophage is derived from monocytes by culturing the latter under appropriate conditions as discussed herein.

The inflammatory condition may be any suitable inflammatory condition in any tissue or organ of the body. Suitably, the inflammatory condition may be present in the liver, lungs, kidney, heart, gastro-intestinal tract, brain, pancreas, thyroid gland, bone or uterine tissue. The inflammatory condition may be acute or chronic. It may be preferred that the condition is chronic. Chronic inflammation may be described as slow, long-term inflammation lasting for prolonged periods of several months to years. The inflammatory condition, whether acute or chronic, may be refractory to conventional treatment.

The inflammatory condition may have a fibrotic element. Fibrosis, or scarring, is defined by the accumulation of excess extracellular matrix components.

Where the engineered macrophage is engineered with exogenous nucleic acid comprising an MMP9 coding sequence, the inflammatory condition may for example be in the lung or liver, preferably in the liver, more preferably wherein the inflammatory condition comprises liver cirrhosis. Where the inflammatory condition is in the lung, the condition may comprise pulmonary fibrosis.

Where the engineered macrophage is engineered with exogenous nucleic acid comprising an MMP12 coding sequence, the inflammatory condition may for example be in the liver, preferably wherein the inflammatory condition comprises liver cirrhosis.

Cirrhosis represents the end-stage of chronic liver injury and progressive fibrosis (scarring), irrespective of the underlying aetiology. It is characterised by severe liver fibrosis leading to architectural disruption, hepatocyte dysfunction and portal hypertension. Various aetiologies may lead to liver cirrhosis. Hepatic disorders having a fibrotic component which may lead to fibrosis include, but are not limited to, non-alcoholic fatty liver disease (NAFL) (e.g., non-alcoholic fatty liver disease (NAFLD) or non-alcoholic steatohepatitis (NASH)) or alcoholic liver disease (e.g., alcoholic fatty liver disease (AFLD) or alcoholic steatohepatitis (ASH)). Fibrotic diseases, disorders, and conditions can include mechanical trauma, biliary obstruction, autoimmune hepatitis, iron overload, Hepatitis B infection (HBV), and/or Hepatitis C infection (HCV). Hepatic disorders having a fibrotic component which may also include, but are not limited to, steatotic liver disease (SLD), which in turn may include metabolic dysfunction-associated steatotic liver disease (MASLD), Metabolic-associated steatohepatitis (MASH), Met-ALD or cryptogenic SLD.

Steatotic liver disease may also be known as fatty liver disease. Metabolic dysfunction-associated steatotic liver disease refers to a non-alcoholic fatty liver disease, and therefore may also be known as NAFLD. Metabolic-associated steatohepatitis refers to a more severe form of MASLD, which may also be known as NASH. “Met-ALD” refers to individuals who have steatotic liver disease and who also drink alcohol. “Cryptogenic SLD” refers to SLD whose cause is unknown, such as in individuals who do not carry any known metabolic risk factors for SLD.

However, the engineered macrophages according to the present invention are able to treat cirrhosis irrespective of the underlying aetiology.

Cirrhosis may be either compensated or decompensated cirrhosis (also referred to herein as hepatic decompensation or HD). Decompensated cirrhosis is defined as an acute deterioration in liver function in a patient with cirrhosis and is characterised by symptoms such as, but not limited to, jaundice, ascites, hepatic encephalopathy, hepatorenal syndrome, variceal haemorrhage, gastrointestinal haemorrhage and any combination thereof. In other embodiments, the liver disease is decompensated cirrhosis. In some embodiments, the liver disease is compensated cirrhosis. In some embodiments, the subject has compensated cirrhosis. In some embodiments, the subject exhibits one or more clinical signs on decompensated cirrhosis selected from the list consisting of jaundice, ascites, hepatic encephalopathy, hepatorenal syndrome, variceal haemorrhage and gastrointestinal haemorrhage.

In some embodiments, the liver disease is one in which cirrhosis is caused by damage to hepatocytes, for example, is a hepatocyte-derived disease, such as those diseases of viral origin (including treated (sustained viral response) hepatitis C (HCV), hepatitis B), damage through alcoholism (alcohol related liver disease (ALD)), or non-alcoholic fatty liver disease (NAFLD), including Non-alcoholic steatohepatitis (NASH) (including NASH resulting from diabetes or obesity), cryptogenic cirrhosis, haemochromotosis or alpha-1-antitrypsin deficiency. In some embodiments, the underlying aetiology has been removed (for example, a patient suffering from damage through alcoholism is no longer drinking, or a patient suffering from damage through HCV no longer has HCV etc.). In some embodiments, the patient with liver disease is at risk of end stage renal disease.

In some embodiments, the liver disease is steatotic liver disease (SLD). In some embodiments, the steatotic liver disease is metabolic dysfunction-associated steatotic liver disease (MASLD), is Met-ALD or cryptogenic SLD. In some embodiments, the metabolic dysfunction-associated steatotic liver disease is Metabolic-associated steatohepatitis (MASH).

Cirrhosis may lead to acute-on-chronic liver failure (ACLF). In some embodiments, the liver disease is ACLF. ACLF is a distinct condition from hepatic decompensation. Hepatic decompensation is characterised by the development of ascites, hepatic encephalopathy, gastrointestinal haemorrhage, or any combination of these conditions in patients with liver cirrhosis. ACLF in contrast is associated with organ failures and carries high short-term mortality in excess of 15% at 28 days. Three major features characterise this syndrome: ACLF occurs in the context of intense systemic inflammation; ACLF frequently develops in close temporal relationship with pro-inflammatory precipitating events (eg infections or alcoholic hepatitis); and ACLF is associated with single- or multiple-organ failure. In some embodiments, the engineered macrophage is used in the treatment of cirrhosis in a subject with ACLF.

A diseased subject suitable for a treatment or use in accordance with any aspect or embodiment of the invention may be a subject with a relevant disease and severity.

The engineered macrophages of the present invention may be for use in therapy. The macrophages of the present invention may be for use in treating an inflammatory condition and/or a fibrotic condition in a subject. As defined above, treating here can mean preventing, reducing or removing inflammation/fibrosis/organ damage. For example, the engineered macrophages maybe administered to a subject at an acute inflammation stage with the aim of preventing a chronic inflammatory condition. The engineered macrophages may also be administered to a subject at a chronic inflammation stage with the aim of preventing/reducing chronic fibrosis. The engineered macrophages may also administered to a subject who is experiencing an acute on chronic inflammatory state, such as acute on chronic liver failure (ACLF).

In some embodiments the condition may be Acute-on-chronic liver failure (ACLF). ACLF is a syndrome characterised by acute decompensation of chronic liver disease associated with organ failures and high short-term mortality. An excessive systemic inflammatory response seems to play a crucial role in the development of ACLF.

In some embodiments, the macrophages that are engineered to express human MMP9 or MMP12 may be used in the treatment of cirrhosis.

In some embodiments, the macrophages that are engineered to express human MMP9 or MMP12 may be used in the treatment of ACLF.

Further optional features are as described in the dependent claims. Any features under any of the sections may be combined with any of the aspects or embodiments of the invention in any workable order.

In certain embodiments, the invention provides:
1. An engineered macrophage for use in treating an inflammatory condition, preferably an inflammatory condition with a fibrotic element, wherein the macrophage is engineered with exogenous nucleic acid encoding MMP9 or MMP12.
2. An engineered macrophage of embodiment 1, wherein said macrophage is derived from a precursor cell.
3. An engineered macrophage of embodiment 1 or embodiment 2, wherein said matrix metalloproteinase is MMP9.
4. An engineered macrophage according to embodiment 3, wherein the inflammatory condition is in the lung or in the liver, preferably in the liver, more preferably wherein the inflammatory condition comprises liver cirrhosis or ACLF.
5. An engineered macrophage of embodiment 1 or embodiment 2, wherein said matrix metalloproteinase is MMP12.
6. An engineered macrophage according to embodiment 5, wherein the inflammatory condition is in the liver, more preferably wherein the inflammatory condition comprises liver cirrhosis or ALCF.
7. An engineered macrophage of any preceding embodiment, wherein said macrophage overexpresses the said coding sequence encoding MMP9 or MMP12.
8. An engineered macrophage of any preceding embodiment, wherein said exogenous nucleic acid comprises a DNA molecule, an RNA molecule, or a non-viral vector.
9. An engineered macrophage of any preceding embodiment, wherein said exogenous nucleic acid is transfected into the macrophage, preferably via electroporation.
10. An engineered macrophage of any preceding embodiment wherein said macrophage has a repressed STING pathway or is treated with anti-inflammatory agents.
11. An engineered macrophage according to any preceding embodiment, wherein said macrophage exhibits a pro-restorative phenotype.
12. An engineered macrophage according to any preceding embodiment, wherein said macrophage is autologous or allogenic to the subject.
13. An engineered macrophage according to any preceding embodiment, wherein said inflammatory condition is chronic.
14. A population of engineered macrophages comprising engineered macrophages for use according to any preceding embodiment.
15. A composition comprising an engineered macrophage or population of engineered macrophages for use according to any preceding embodiment.
16. A method of improving the resolution of fibrosis in a chronic condition, comprising the use of the engineered macrophage, population of engineered macrophages or a composition according to any of the preceding embodiments.
17. A method of treating an inflammatory condition, preferably an inflammatory condition with a fibrotic element, comprising the use of a macrophage engineered with an exogenous sequence encoding for MMP9 or MMP12.

The invention also provides the following embodiments, which may be combined with any other embodiments:

1. An engineered macrophage for use in treating an inflammatory condition with a fibrotic element, wherein the macrophage is engineered with exogenous nucleic acid encoding MMP9.

2. An engineered macrophage according to embodiment 1, wherein said macrophage is derived from a precursor cell.

3. An engineered macrophage according to embodiment 1 or 2, wherein the inflammatory condition is in the lung or in the liver, preferably in the liver, more preferably wherein the inflammatory condition comprises liver cirrhosis or ACLF.

4. An engineered macrophage according to embodiment 3, wherein the inflammatory condition comprises liver cirrhosis.

5. An engineered macrophage according to embodiment 4, wherein the liver cirrhosis resulted from at least one disease or condition selected from the group consisting of: non-alcoholic fatty liver disease (NAFL) (e.g., non-alcoholic fatty liver disease (NAFLD) or non-alcoholic steatohepatitis (NASH)), alcoholic liver disease (e.g., alcoholic fatty liver disease (AFLD) or alcoholic steatohepatitis (ASH)), mechanical trauma to the liver, biliary obstruction, autoimmune hepatitis, iron overload, Hepatitis B infection (HBV) and Hepatitis C infection (HCV).

6. An engineered macrophage according to embodiment 4 wherein the liver cirrhosis resulted from steatotic liver disease (SLD), optionally wherein the steatotic liver disease is metabolic dysfunction-associated steatotic liver disease (MASLD), metabolic-associated steatohepatitis (MASH), Met-ALD or Cryptogenic SLD.

7. An engineered macrophage according to any of embodiments 4-6, wherein the liver cirrhosis is selected from compensated cirrhosis and decompensated cirrhosis.

8. An engineered macrophage according to embodiment 7, wherein the cirrhosis is decompensated cirrhosis, and the subject to be treated exhibits one or more clinical signs of decompensated cirrhosis selected from the list consisting of jaundice, ascites, hepatic encephalopathy, hepatorenal syndrome, variceal haemorrhage and gastrointestinal haemorrhage.

9. An engineered macrophage according to any preceding embodiment, wherein said macrophage overexpresses the said coding sequence encoding MMP9.

10. An engineered macrophage according to any preceding embodiment, wherein said exogenous nucleic acid comprises a DNA molecule, an RNA molecule, or a non-viral vector.

11. An engineered macrophage according to any preceding embodiment, wherein said exogenous nucleic acid is transfected into the macrophage, preferably via electroporation.

12. An engineered macrophage according to any preceding embodiment, wherein said macrophage has a repressed STING pathway or is treated with anti-inflammatory agents.

13. An engineered macrophage according to any preceding embodiment, wherein said macrophage exhibits a pro-restorative phenotype.

14. An engineered macrophage according to any preceding embodiment, wherein said macrophage is autologous or allogenic to the subject, optionally wherein the macrophage is derived from a human monocyte, or a stem cell, further optionally wherein the stem cell is an induced pluripotent stem cell.

15. An engineered macrophage according to any preceding embodiment, wherein said inflammatory condition is chronic.

16. An engineered macrophages according to any preceding embodiment, wherein the engineered macrophage secretes MMP9 and the secreted MMP9 protein level is greater than 200 ng/ml when the macrophage is cultured in vitro at a cell concentration of 4×10⁶/ml.

17. A population of engineered macrophages comprising engineered macrophages for use according to any preceding embodiment.

18. A composition comprising an engineered macrophage or population of engineered macrophages for use according to any preceding embodiment.

19. A method of improving the resolution of fibrosis in a chronic condition, comprising the use of an engineered macrophage, population of engineered macrophages or a composition according to any of the preceding embodiments.

20. A method of treating an inflammatory condition with a fibrotic element, comprising the use of a macrophage engineered with an exogenous sequence encoding for MMP9.

21. An engineered macrophage, wherein the macrophage is engineered to express MMP9.

22. The engineered macrophage of embodiment 21, wherein the engineered macrophage comprises one or more exogenous coding sequences for MMP9.

23. An engineered macrophage of embodiment 22, wherein said exogenous nucleic acid is transfected into the macrophage, optionally via electroporation.

24. The engineered macrophage of embodiment 22 or 23, wherein the macrophage is transfected with a DNA vector, an RNA vector optionally mRNA, a vector not derived from a viral genome or one or more free nucleic acids.

25. The engineered macrophage of any of embodiments 21-24, wherein the MMP9 protein expressed is at least 85%, at least 90%, at least 95%, 96%, 97%, 98%, 99% homologous or similar to the sequence presented as SEQ ID No. 2.

26. The engineered macrophage of any one of embodiments 21-25, wherein the sequence of the MMP9 coding sequence provided is at least 85%, at least 90%, at least 95%, 96%, 97%, 98%, 99% homologous or similar to SEQ ID NO: 1.

27. The engineered macrophage of any one of embodiments 21-25, wherein the sequence of the MMP9 coding sequence provided is at least 80%, at least 85%, at least 90%, at least 95%, at least 96%, at least 97%, at least 98%, or at least 99% identical to SEQ ID No. 5.

28. The engineered macrophage of embodiment 21, wherein the macrophage is engineered to turn on or upregulate endogenous genes that encode MMP9.

29. The engineered macrophage of embodiment 21 or 28, wherein the macrophage is engineered to turn off or downregulate endogenous genes which downregulate MMP9 expression.

30. The engineered macrophage of any one of embodiments 21-29, wherein the macrophage is engineered to overexpress MMP9.

31. The engineered macrophage of any one of embodiments 21-30, wherein the macrophage is derived from a human monocyte or a stem cell, optionally an induced pluripotent stem cell.

32. An engineered macrophage of any of embodiments 21-31, wherein said macrophage has a repressed STING pathway or is treated with anti-inflammatory agents.

33. An engineered macrophage of any of embodiments 21-32, wherein said macrophage exhibits a pro-restorative phenotype.

34. An engineered macrophage of any of embodiments 21-33, wherein the engineered macrophage secretes MMP9 and the secreted MMP9 protein level is greater than 200 ng/ml when cultured in vitro at a cell concentration of 4×10⁶/ml.

35. A population of engineered macrophages comprising engineered macrophages according to any of embodiments 21-34.

36. A composition comprising an engineered macrophage of any of embodiments 21-34, or population of engineered macrophages according to embodiment 35.

37. An engineered macrophage, population of engineered macrophages or a composition according to any of embodiments 21-36 for use in treating an inflammatory condition, preferably an inflammatory condition with a fibrotic element, preferably liver cirrhosis.

38. A method of improving the resolution of fibrosis in a chronic condition, comprising the use of an engineered macrophage, population of engineered macrophages or a composition according to any of embodiments 21-36.

Further advantages are described below.

### BRIEF DESCRIPTION OF THE DRAWINGS

The present invention will be described further below with reference to exemplary embodiments and the accompanying drawings, in which:

FIG. 1—(A,B): Expression levels of MMP9 (A) and MMP12 (B) in macrophages transfected respectively with MMP9 or MMP12-encoding plasmids, as determined by qPCR of mRNA levels normalised with respect to PIK3C2A; (C,D): expression levels of MMP9 (C) and MMP12 (D) protein in cell culture supernatants were also determined, using ELISA. Expression is measured 24h post-transfection. NT=Non-transfected; NT+STINGi=non-transfected+STING inhibition cocktail (rIL4+rIL13). MMPx Trx=transfected with MMPx.

FIG. 2—Total MMP activity measured in cell culture supernatants of various populations of hMDMs by fluorescence emission.

FIG. 3—Heat map representation of flow Cytometry analysis of cell surface marker expression ((A) CD14, (B) CD206, (C) 25F9) in non-transfected (NT), NT cultured in the presence of the STING inhibitors cocktail (STINGi), MMP9-transfected (MMP9 Trx) and MMP12-transfected (MMP12 Trx) hMDMs 24h post-transfection. Each row represents a distinct donor. Mean Fluorescence Intensity (MFI) values have been used to generate the heat map (darker=lower MFI; lighter=higher MFI).

FIG. 4—(A) Dosage of multiple cytokines in the cell culture supernatant of non-transfected (NT), NT+Sting inhibitor cocktail (NT+STINGi), CCR2, MMP9 and MMP12 transfected (Trx) hMDMs. (B) Dosage of multiple MMPs in the cell culture supernatant of NT, NT+STINGi and MMP9-Trx hMDMs. (A-B). Values used to create the heat map are average of at least four donors/group. Black is the highest expression for each analyte, and light grey is the minimal expression for each analyte.

FIG. 5—Percentage (%) of phagocytosing hMDMs measured by flow cytometry after 1h incubation with E. coli-coated, pH-sensitive beads.

### DETAILED DESCRIPTION OF THE INVENTION

The present invention relates to an engineered macrophage engineered with a coding sequence for at least one matrix metalloproteinase (MMP), preferably MMP9 or MMP12. The resulting engineered macrophage has desirable anti-fibrotic properties making it suitable for use in treating inflammatory condition which may have a fibrotic element.

A ‘macrophage’ as used herein refers to a phagocytic cell which is responsible for detecting, engulfing and destroying pathogens and apoptotic cells. A macrophage may be produced through the differentiation of any suitable precursor cell, including monocytes. An ‘engineered macrophage’ of the present invention is a macrophage that has been engineered to overexpress one or more matrix metalloproteinases (MMPs).

### Matrix Metalloproteinases

Matrix metalloproteinases (MMPs), also known as matrix metalloproteinases or matrixins are a family of peptidases, collectively capable of cleaving all components of the extracellular matrix (ECM). MMPs are also able to process bioactive mediators, such as growth factors, cytokines, chemokines, and cell-surface receptors. 25 mammalian MMPs have been identified (Table 1), with varying roles in the maintenance of the ECM and processes of tissue repair, and both inhibitory and stimulatory roles in fibrosis.

TABLE 1
Mammalian MMPs

| Gene | Name(s) |
|---|---|
| MMP1 | Interstitial collagenase, CLG, CLGN |
| MMP2 | Gelatinase-A, 72 kDa gelatinase |
| MMP3 | Stromelysin 1, Stromelysin 1 |
| MMP7 | Matrilysin, PUMP 1, Matrilysin, PUMP 1 |
| MMP8 | Neutrophil collagenase, CLG1, HNC, MMP-8, PMNL-CL |
| MMP9 | Gelatinase-B, 92 kDa gelatinase, CLG4B, GELB, MANDP2, MMP-9, matrix metallopeptidase 9 |
| MMP10 | Stromelysin 2, SL-2, STMY2 |
| MMP11 | Stromelysin 3, SL-3, ST3, STMY3 |
| MMP12 | Macrophage metalloelastase, Macrophage elastase, HME, ME, MME, MMP-12, matrix metallopeptidase 12 |
| MMP13 | Collagenase 3, CLG3, MANDP1, MMP-13 |
| MMP14 | MT1-MMP, MMP-14, MMP-X1, MT-MMP, MT-MMP 1, MT1-MMP, MT1MMP, MTMMP1, WNCHRS |
| MMP15 | MT2-MMP, MT2-MMP, MTMMP2, SMCP-2, MMP-15, MT2MMP |
| MMP16 | MT3-MMP, C8orf57, MMP-X2, MT-MMP2, MT-MMP3, MT3-MMP |
| MMP17 | MT4-MMP, MT4-MMP, MMP-17, MT4MMP, MTMMP4 |
| MMP18 | Collagenase 4, xcol4, xenopus collagenase |
| MMP19 | RASI-1, stromelysin-4, RASI-1, CODA |
| MMP20 | Enamelysin, AI2A2, MMP-20 |
| MMP21 | X-MMP, MMP-21, HTX7 |
| MMP23A | CA-MMP |
| MMP23B | MIFR, MIFR-1, MMP22, MMP23A |
| MMP24 | MT5-MMP, MMP-24, MMP25, MT-MMP 5, MT-MMP5, MT5-MMP, MT5MMP, MTMMP5 |
| MMP25 | MT6-MMP, MMP-25, MMP20, MMP20A, MMPL1, MT-MMP 6, MT-MMP6, MT6-MMP, MT6MMP, MTMMP6 |
| MMP26 | Matrilysin-2, endometase |
| MMP27 | MMP-22, C-MMP, MMP-27 |
| MMP28 | Epilysin, MM28, MMP-25, MMP-28, MMP25 |

MMP9 (Matrix metallopeptidase 9) is a matrix metalloproteinase, a type IV collagenase. MMP9 is also known as 92 kDa type IV collagenase, 92 kDa gelatinase or gelatinase B (GELB).

In some embodiments, the MMP9 is human MMP9 as described in GenBank accession number NM_004994.3. In some embodiments, the human MMP9 comprises the amino acid sequence of SEQ ID NO. 2. In some embodiments, the amino acid sequence is encoded by an mRNA comprising the sequence of SEQ ID NO. 1 or SEQ ID NO: 5.

MMP12 (Matrix metallopeptidase 12) is a matrix metalloproteinase, and degrades soluble and insoluble elastin. MMP12 is also known as macrophage metalloelastase (MME) or macrophage elastase (ME).

In some embodiments, the MMP12 is human MMP12 as described in GenBank accession number NM_002426.6. In some embodiments, the human MMP12 comprises the amino acid sequence of SEQ ID NO. 4. In some embodiments, the amino acid sequence is encoded by an mRNA comprising the sequence of SEQ ID NO. 3.

A macrophage may be transfected with an exogenous nucleic acid comprising a coding sequence encoding MMP9 or MMP12 for use in treating an inflammatory condition with a fibrotic element.

### Engineered Macrophages

The macrophages may be transfected with one or more exogenous nucleic acid constructs, including one or more coding sequences encoding an MMP (MMP9 or MMP12). The construct may be any suitable construct, but is preferably a DNA construct or an RNA construct.

The macrophage engineered to express MMP9 or MMP12 may be genetically engineered in any suitable way. For example, using viral or non-viral vectors, DNA or RNA constructs or gene editing using any suitable technology. Thus, in some embodiments, the macrophage may be engineered with one or more exogenous coding sequences. These exogenous coding sequences may encode MMP9 and/or MMP12 and/or they may encode gene editing proteins such as CRISPR, nickases, and the like, that are capable of altering the cell's genome to increase the expression of endogenous MMP9 and/or endogenous MMP12. This could be achieved by editing the promoter or enhancer sequence, for example.

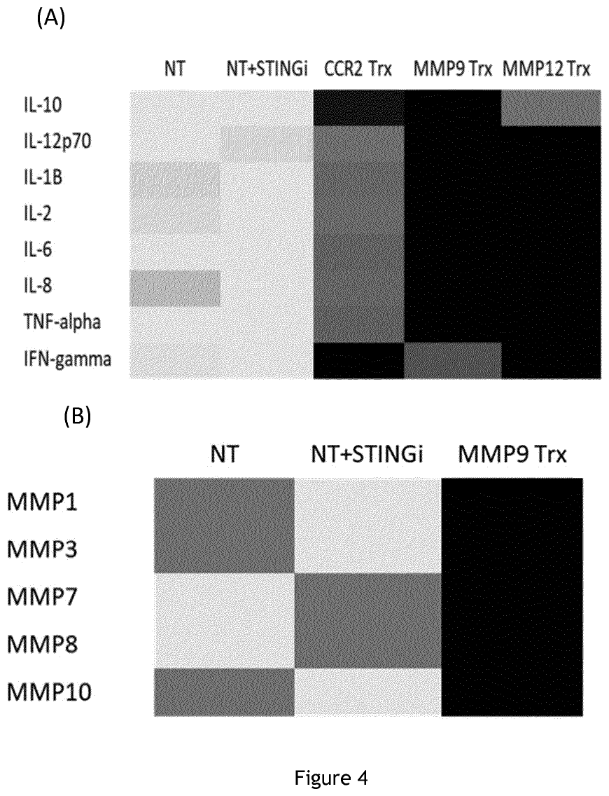

The inventors have found that transfecting macrophages with an MMP9-expressing construct or MMP12-expressing construct achieves upregulation and secretion of active MMPs without the upregulation of TIMPs, which was surprising. They have also found that transfecting macrophages with an MMP-expressing construct lead to co-upregulation of other MMPs beyond the transfected MMP of choice (see Examples and FIG. 4B). This latter result was particularly surprising. Both of these qualities of macrophages of the present invention would be expected to be beneficial in an anti-fibrotic treatment. Thus, by engineering a macrophage with either MMP9 or MMP12, the activity of a plurality of MMPs are increased.

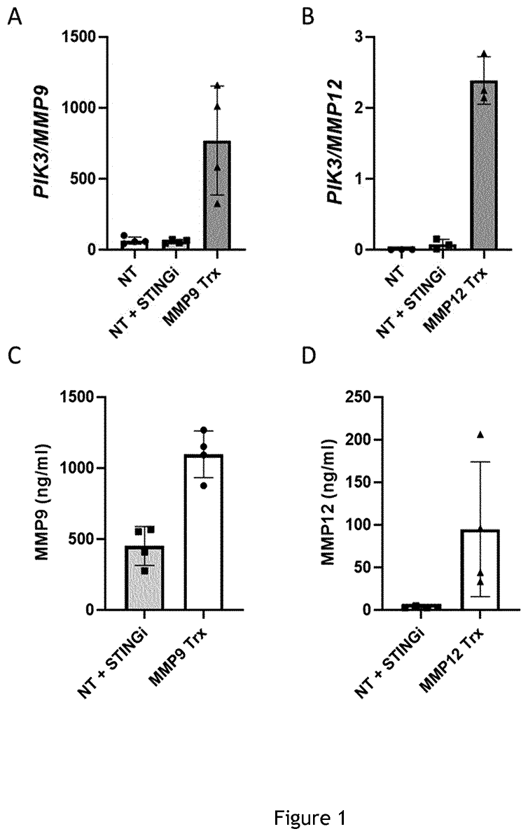

The results herein show that transfecting macrophages with an MMP9-encoding plasmid induces a significant increase in MMP9 transcription within 24 hours compared to non-transfected macrophages (FIG. 1A). Furthermore, secreted MMP9 protein levels were also significantly increased as compared to non-transfected controls (FIG. 1C). This evidence suggests that the electroporation method employed efficiently transfects macrophages with an MMP9-encoding plasmid, and this results in a rapid, reliable, and robust increase in MMP9 at both the transcript and protein level.

The results herein also show that transfecting macrophages with an MMP12-encoding construct induces a significant increase in MMP12 transcription within 24 hours compared to non-transfected macrophages (FIG. 1B). Furthermore, secreted MMP12 protein levels were also significantly increased as compared to non-transfected controls (FIG. 1D). This evidence suggests that the electroporation method employed efficiently transfects macrophages with an MMP12-encoding plasmid, and this results in a rapid, reliable, and robust increase in MMP12 at both the transcript and protein level.

Suitably, the engineered macrophage may be non-virally engineered. Suitably the macrophage may be transfected with a DNA vector. Suitably the DNA vector may be a naked DNA vector, such that it is not associated with proteins and/or lipids. Suitably the DNA vector may not be derived from a viral genome. The DNA vector may be an episomal vector such that it can function without integrating into the chromosomes of the macrophage.

The DNA vector may comprise at least one sequence encoding MMP9 and/or MMP12.

The DNA vector may comprise at least one sequence encoding MMP9 and/or MMP12, operably linked to a promoter.

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of affecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the DNA sequence of interest which allows for initiation of transcription of the DNA sequence of interest upon recognition of the promoter element by a transcription complex. In one embodiment, the DNA vector may include one or more liver specific promoters or cirrhosis specific promoters. In some embodiments, the DNA vector may comprise a CX3CR1 promoter, an insulin-like growth factor 1 (IGF1), or a CD11B promoter.

Suitably, the macrophage is a genetically engineered macrophage comprising an extrachromosomal/episomal construct overexpressing MMP9 and/or MMP12.

Suitably, the macrophage may be transfected with exogenous RNA, preferably exogenous mRNA, encoding MMP9 and/or MMP12.

The engineered macrophage may be transfected with any suitable nucleic acid via electroporation.

Other suitable methods of transfection include nucleofection.

The engineered macrophage may be transfected with one or more free nucleic acids or vectors.

The engineered macrophage may be engineered to overexpress MMP9 and/or MMP12.

### Properties of Engineered Macrophages

The inventors have found that transfecting macrophages with an MMP9-encoding construct or MMP12-encoding construct achieves upregulation and secretion of a plurality of active MMPs without the upregulation of TIMPs. They also found the transfected macrophages displayed co-upregulation of other MMPs beyond the exogenously-encoded MMP (see Examples and FIG. 4B). These are favourable characteristics for an engineered macrophage for use in treating an inflammatory condition with a fibrotic element.

### TIMPS

When a cell overexpresses MMPs it can upregulate its inhibitors, the Tissue Inhibitors of Metalloproteinases (TIMPs). The upregulation of TIMPs would make the MMPs non-functional.

The inventors have demonstrated that MMP-transfected macrophages have sustained MMP activity, suggesting upregulation of functional MMPs without the upregulation of TIMPs.

Suitably, the transfected engineered macrophage of the present invention may overexpress the transfected MMP, with minimal or no upregulation of TIMPs.

### Co-regulation of Other MMPs

Most surprisingly, the inventors have found that MMP-transfected engineered macrophages are able to co-upregulate other MMPs beyond the MMP introduced through transfection. FIG. 4B shows that MMP9-transfected macrophages strongly upregulate several other MMPs, such as MMP1, MMP8 and MMP10. Thus a plurality of other MMPs are affected. Co-upregulation of these further MMPs could contribute significantly to the anti-fibrotic effect of the macrophage cell therapy of the present invention. For example, MMP8 is normally contained in neutrophils granules and digests collagen I, II, and III, thereby being of particular interest for liver fibrosis²⁷, where most of the collagen in the fibrotic septa is collagen I and III²⁸.

Suitably, the transfected engineered macrophage of the present invention may upregulate expression of a plurality of other MMPs beyond the MMP that is introduced through transfection.

### Engineered Macrophages for Use in Treating an Inflammatory Condition

Suitably, the invention relates to a cell therapy product for inflammatory organ damage based on macrophages genetically modified with exogenous nucleic acids that increase their anti-fibrotic abilities. Suitably, the engineered macrophage has a pro-restorative phenotype and is anti-inflammatory and anti-fibrotic.

In some embodiments, a pro-restorative phenotype may be described using one or more of the following markers: an increase in CD206 and/or CD163, a decrease in inflammatory markers such as CD86 and/or MHC class II (HLA-DR). These increases/decreases are as compared to non-polarised (resting) or pro-inflammatory macrophages. In terms of secretion profile, these macrophages are expected not to express TNFa, IFNg and IL1b, normally associated with a pro-inflammatory and pro-fibrotic profile.

‘Treatment’ as used in the present invention means an intervention in a physiological condition which prevents, reduces, or removes the clinical symptoms associated with a given physiological condition in a subject.

By ‘subject’ or ‘individual’ or ‘animal’ or ‘patient’ is meant any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on.

Suitably the subject may be in need of treatment. Suitably therefore the subject may have a disease, or be at risk of developing a disease. Suitably the subject may display one or more symptoms of a disease.

The engineered macrophages of the present invention are for use in treating an inflammatory condition, preferably one that has or may develop a fibrotic element in a subject. As defined above, treating here can mean preventing, reducing or removing inflammation and/or fibrosis. For example, the engineered macrophage may be administered to a subject at an acute inflammation stage with the aim of preventing a chronic inflammatory condition with a fibrotic element. The engineered macrophage may also be administered to a subject at a chronic inflammation stage with the aim of preventing/reducing chronic fibrosis. In preferred embodiments, the chronic inflammatory liver injury with a fibrotic element is liver cirrhosis. In some embodiments, the liver disease is decompensated cirrhosis. In some embodiments, the liver disease is compensated cirrhosis.

In some embodiments, the condition is decompensated liver cirrhosis and the subject exhibits one or more clinical signs of hepatic decompensation selected from the list consisting of jaundice, ascites, hepatic encephalopathy, hepatorenal syndrome, variceal haemorrhage and gastrointestinal haemorrhage.

In some embodiments, the liver disease is one in which cirrhosis is caused by damage to hepatocytes, for example, is a hepatocyte-derived disease, such as those diseases of viral origin (including treated (sustained viral response) hepatitis C (HCV), hepatitis B), damage through alcoholism (alcohol related liver disease (ALD)), or non-alcoholic fatty liver disease (NAFLD), including Non-alcoholic steatohepatitis (NASH) (including NASH resulting from diabetes or obesity), cryptogenic cirrhosis, haemochromotosis or alpha-1-antitrypsin deficiency. In some embodiments, the underlying aetiology has been removed (for example, a patient suffering from damage through alcoholism is no longer drinking, or a patient suffering from damage through HCV no longer has HCV etc.). In some embodiments, the patient with liver disease is at risk of end stage renal disease.

In some embodiments, the liver disease is steatotic liver disease (SLD). In some embodiments, the steatotic liver disease is metabolic dysfunction-associated steatotic liver disease (MASLD), is Met-ALD or cryptogenic SLD. In some embodiments, the metabolic dysfunction-associated steatotic liver disease (MASLD) is Metabolic-associated steatohepatitis (MASH).

Metabolic dysfunction-associated steatotic liver disease refers to a non-alcoholic fatty liver disease, and therefore may also be known as NAFLD. Metabolic-associated steatohepatitis refers to a more severe form of MASLD, which may also be known as NASH. “Met-ALD” refers to individuals who have steatotic liver disease and who also drink alcohol. “Cryptogenic SLD” refers to SLD whose cause is unknown, such as in individuals who do not carry any known metabolic risk factors for SLD.

Suitably an acute disease or injury may be classed as a disease or injury with an onset of less than 24 weeks from cause. Suitably a chronic disease may be classed as a disease or injury which has persisted for more than 6 months. Suitably an acute-on chronic disease may be classed as a disease or injury with an onset of less than 24 weeks from cause in a patient that already has a chronic disease that has persisted for more than 6 months. Suitably, engineered macrophages of the present invention may be administered to a subject with an acute occurrence to prevent transition to or increase of chronic inflammation and fibrosis.

In some embodiments, the condition is in or affects an organ selected from the group consisting of: a liver, lung, heart, kidney, pancreas, skin, gastrointestinal, bone marrow, hematopoietic tissue, nervous system, eye or a combination thereof.

In some embodiments the condition may be Acute-on-chronic liver failure (ACLF). ACLF is a syndrome characterised by acute decompensation of chronic liver disease associated with organ failures and high short-term mortality. An excessive systemic inflammatory response seems to play a crucial role in the development of ACLF. Suitably, the condition is chronic organ damage associated with chronic inflammation. Suitably, the condition relates to the kidney, liver, or lung. For example, the condition maybe inflammatory liver damage, inflammatory kidney damage or inflammatory lung damage.

Suitably, the macrophage is autologous or allogenic to the subject.

The invention also relates to a population of engineered macrophages for use as a cell therapy.

Suitably, the use comprises administering an effective amount of the engineered macrophage to the subject.

The invention also relates to a composition comprising the engineered macrophages of the invention or a population thereof. Suitably, the composition is a pharmaceutical composition.

Suitably, the engineered macrophages may be formulated into a pharmaceutical composition.

Suitably the composition is suitable for administration to a subject. Suitably the composition is a liquid.

Suitably the composition is an infusible liquid.

Suitably, delivery to a subject is by systemic administration, suitably by systemic injection.

Suitably the engineered macrophages are for administration to a subject by any route. Suitably the engineered macrophages are for administration to a subject by infusion. Suitably the engineered macrophages are for administration to a subject parenterally, suitably intravenously. Suitably the engineered macrophages are for administration to a subject by injection or infusion. Suitably the engineered macrophages are for administration to a subject intravenously by infusion.

Suitably, administration may be by local injection, e.g. for kidney or liver. Suitably, administration may be by nebulizer, e.g. for lung.

It is preferred that the macrophage for use in therapy is engineered ex vivo and delivered to the patient.

However, in some embodiments, the macrophage may be engineered in vivo. In some embodiments, the macrophage is engineered in vivo via the administration to a subject of a preparation of exogenous coding sequence for MMP9 or MMP12 suitable for transfecting a macrophage. The preparation may include any of the exogenous coding sequences discussed herein. Suitable delivery vehicles for in vivo engineering may include targeting molecules for macrophages by virtue of their cell surface markers. Should an in vivo transfection of macrophages be envisaged, localized application of the preparation may be more effective, such as localized injection of the preparation to liver or kidneys, or nebulization to the lungs.

Suitably, the invention relates to a cell therapy product for inflammatory organ damage based on monocyte-derived macrophages genetically modified with payloads that induce a pro-restorative phenotype.

The M2-like phenotype is pro-restorative, whereas the M1-like phenotype is pro-inflammatory.

### Producing an Engineered Macrophage Overexpressing a Matrix Metalloproteinase

The invention also relates to a method of producing an engineered macrophage overexpressing one or more MMPs. The invention also relates to a method of producing an engineered macrophage overexpressing MMP9 and/or MMP12 comprising transfecting a macrophage with an exogenous nucleic acid comprising at least one sequence encoding MMP9 and/or MMP12 and optionally contacting the macrophage with one or more STING inhibitors/anti-inflammatory treatments. Suitably the engineered macrophages produced can be used for cell therapy.

Suitably, the engineered macrophages produced are manufactured to a GMP-compliant standard. Suitably therefore the engineered macrophages and populations thereof are GMP-compliant.

### Macrophages for Transfection

The macrophages for transfection may be isolated from a human, preferably the subject. The macrophages may be produced from any suitable progenitor cell. Suitable progenitor cells include hematopoietic cells. Preferably, the precursor cell is a monocyte. Suitably the macrophages have been produced in vitro.

The macrophages for transfection may be monocyte-derived. The macrophages for transfection may be human monocyte derived macrophages (hMDM). Monocyte-derived refers to macrophages differentiated from monocytes. Monocytes are the natural precursors of macrophages and dendritic cells; they are contained in blood and bone marrow. Suitably the macrophages are derived from peripheral blood monocytes, suitably the macrophages are peripheral blood monocyte-derived macrophages. Suitably the macrophages are human peripheral blood monocyte derived macrophages. Suitably the monocytes are isolated from a human subject.

The macrophages may be derived from the monocytes by culturing the monocytes, suitably in vitro. The macrophages may be derived from the monocytes by using any suitable culturing method.

The macrophages may be produced in vitro from monocytes by a culturing method lasting between 3 to 8 days optionally 4 to 8 days. Suitably the macrophages are produced in vitro from monocytes by a culturing method lasting between 3 to 7 days, notably 4 to 7 days, or 5 to 7 days. In one embodiment, the macrophages are produced in vitro from monocytes by a culturing method that lasts 3-5 days, 4 or 5 days, or 7 days, known as a day5 method or a day7 method, respectively. One example of an in vitro method of producing macrophages from monocytes is described in WO2019/175595. The ‘day5’ method is described in application WO2021/240167 (the contents of which is herein incorporated by reference).

The macrophages may be produced by a ‘day 5’ method comprising:
(a) Culturing monocytes in medium for 3 to 5 or 4 to 5 days to produce macrophages, wherein the medium comprises one or more growth factors to stimulate macrophage production;
wherein step (a) takes place entirely in the same medium.

The medium may comprise one or more growth factors selected from the CSF family, preferably M-CSF. The medium may contain M-CSF at a concentration of between 25-150 ng/mL. The medium may contain 100 ng/mL GMP-graded recombinant human macrophage colony-stimulating factor 1 (rhM-CSF-1; also known as ‘rh (recombinant human) CSF-1’).

### Transfection

The macrophages may be transfected with an exogenous nucleic acid and further may be contacted with one or more STING inhibitors or indeed anti-inflammatory treatment at a suitable point in the transfection protocol.

Suitable exogenous nucleic acids may be DNA vectors, preferably non-viral DNA vectors. Suitable vectors include DNA constructs e.g. episomal constructs such as plasmids. Suitably the exogenous nucleic acid encodes one or more MMP and a promoter. Suitably the exogenous nucleic acid encodes all sequences necessary and sufficient for transient overexpression of the MMP by the macrophage.

Suitably the encoded MMP protein may be secreted by the macrophage. Suitably the promoter is a constitutive promoter. Suitably the MMP is either or both of MMP9 and MMP12.

The method of transfection may be a method of nucleofection of macrophages in which the nucleic acid enters the nucleus of the cell. The method of nucleofection may comprise a lipid-nucleic acid complex being transferred via electroporation in the cells.

The method of transfection may comprise electroporation. Typically, electroporation comprises the application of an electric current to cells by the use of electrodes. Typically, the cells are placed between the two electrodes before pulses of current are generated across the cells. The electrical pulses induce the temporary formation of pores in the cell membrane so that the nucleic acid can enter the cell through the pores by passive diffusion, or by active electrophoretic motion induced by the electric field if the nucleic acid is charged.

The electroporation steps may be performed by using any electroporator apparatus and programming the electroporator with the desired electroporation conditions. The electroporation steps may be performed by using a cliniMACS electroporator (Miltenyi) or other suitable electroporator. The electroporation steps may be performed by using a Lonza GMP Amaxa.

Prior to electroporation, the macrophages may be contacted with the nucleic acid (NA). The macrophages may be contacted with the NA in solution. Suitably the solution is conductive. The solution may be an electroporation solution. The electroporation solution may be a buffer suitable for use with the chosen electroporator.

Suitably, pre-electroporation and post-electroporation, plating cells comprises plating 2×10⁶ hMDMs/cm² at 4×10⁶/mL.

At the point of electroporation, cell density may range from 50×10⁶ cells/mL to 150×10⁶ cells/mL. At the point of electroporation, macrophages may be present in the solution at a cell density of at least 5×10⁷ cells/mL.

Suitable transfection methods, comprising suitable electroporation methods, for producing a macrophage expressing a protein of interest are described in WO2021/240167. Other suitable electroporation methods may be used.

The method of transfection according to the invention may comprise the steps of:
(a) Contacting a macrophage with a DNA vector comprising at least one sequence encoding an MMP;
(b) Electroporating the macrophage with a first pulse phase, wherein the first pulse phase comprises a burst of unipolar pulses or a square pulse, wherein each pulse is between 750-1000V, and wherein the first pulse phase lasts for a total period of between 20-500 μs;
(c) Electroporating the macrophage with a second pulse phase, wherein the second pulse phase comprises a burst of pulses or a square pulse, wherein each pulse is between 50-225V, and wherein the second pulse phase lasts for a total period of 2000-50000 μs.

The MMP may be MMP9 and/or MMP12.

The method of producing an engineered macrophage overexpressing an MMP may comprise the steps of:
(a) Contacting a macrophage with a DNA vector comprising at least one sequence encoding an MMP;
(b) Electroporating the macrophage; and
(c) Contacting the macrophage with one or more STING inhibitors.

The MMP may be MMP9 and/or MMP12.

Suitably, the method of producing an engineered macrophage overexpressing an MMP may comprise the steps of:
(a) Contacting a macrophage with a DNA vector comprising at least one sequence encoding an MMP;
(b) Electroporating the macrophage with a first pulse phase, wherein the first pulse phase comprises a burst of unipolar pulses or a square pulse, wherein each pulse is between 750-1000V, and wherein the first pulse phase lasts for a total period of between 20-500 μs;
(c) Electroporating the macrophage with a second pulse phase, wherein the second pulse phase comprises a burst of pulses or a square pulse, wherein each pulse is between 50-225V, and wherein the second pulse phase lasts for a total period of 2000-50000 μs; and
(d) Contacting the macrophage with one or more STING inhibitors.

The MMP may be MMP9 and/or MMP12.

### STING Inhibitor/Anti-Inflammatory Treatment

The method of producing an engineered macrophage according to the invention comprises transfecting a macrophage with an exogenous nucleic acid, optionally a nucleic acid vector such as a DNA vector or RNA vector, comprising at least one sequence encoding the relevant MMP, and contacting the macrophage with one or more Stimulator of Interferon Genes (STING) inhibitors or anti-inflammatory treatments.

A STING inhibitor is a STING pathway inhibitor. Suitably the STING pathway inhibitor represses the STING pathway. Suitably repression of the STING pathway prevents activation of interferon secretion. Some STING inhibitors are also anti-inflammatory.

The STING pathway inhibitor may be added during the method of transfecting the macrophages.

The STING pathway inhibitor may be added after the electroporation steps. Therefore the method may comprise a step of contacting the macrophages with a STING pathway inhibitor, suitably contacting the transfected macrophages with a STING pathway inhibitor.

The method may comprise the steps of:
(a) Contacting a engineered macrophage with a DNA vector comprising at least one sequence encoding an MMP (MMP9 or MMP12);
(b) Electroporating the macrophage; and
(c) Contacting the macrophage with one or more STING inhibitors.

The MMP may be MMP9 and/or MMP12.

STING pathway inhibition or repression may be achieved by contacting the macrophage after transfection with anti-inflammatory cytokines. Notably, the transfected macrophage may be contacted by IL10, IL4 and IL13 (IL4 and IL13). Preferably the STING inhibitor cocktail comprises IL4 and IL13. This provides a good balance between repression of the STING pathway and allowing effective overexpression of the one or more MMPs in the transfected macrophage.

The transfected macrophages may be contacted with these anti-inflammatory cytokines (IL10 and/or IL4/IL13) for a period of about 2 hours to about 48 hours, suitably 4 hours to 40 hours, suitably 12 to 24 hours, optionally around 16 hours.

The transfected macrophages may be contacted with these anti-inflammatory cytokines (1L4/1L13) at a concentration of between 2 ng/mL and 200 ng/mL, suitably between 5 ng/mL and 150 ng/mL, suitably between 10 ng/mL to 100 ng/mL, suitably between 15 ng/mL to 75 ng/mL, suitably between 20 ng/mL to 50 ng/mL.

Alternatively, the STING pathway inhibitor may be a small molecule. Suitably the STING inhibitor may be selected from BX-795, H-151, Amlexanox, MRT67307, for example.

Suitably the STING pathway inhibitor is used as a solution.

For the step of contacting the macrophage with one or more STING inhibitors, the cells may be plated as follows: 2×10⁶ macrophages/cm² at 4×10⁶/mL.

Repression of the STING pathway may be achieved by inhibiting or repressing one or more elements of the STING pathway, and/or one or more elements controlling the STING pathway. Such inhibition or repression of the STING pathway may comprise inhibiting or blocking the activation of the STING pathway, suitably therefore inhibition or repression of the STING pathway may comprise non-activation of the STING pathway.

Suitably the STING pathway may be considered as repressed or inhibited. Suitably the STING pathway is repressed or inhibited relative to a transfected macrophage that is not contacted with, or does not comprise a heterologous nucleic acid encoding, a STING pathway inhibitor. The STING pathway may be repressed by at least 10%, at least 15%, at least 20%, at least 25%, at least 30%, at least 35%, at least 40%, at least 45%, at least 50%, at least 55%, at least 60%, at least 65%, at least 70%, at least 75% relative to a transfected macrophage that is not contacted with, or does not comprise a heterologous nucleic acid encoding, a STING pathway inhibitor.

### The Following Definitions are Provided

‘payload’ as used herein is a gene or genes of therapeutic interest, which is introduced via transfection to test its effect on macrophages.
‘non-polarized macrophage’ as used herein refers to a mature macrophage which has not received any further stimulation to induce particular functional capacity, non-polarized macrophages may also refer to naïve, resting or non-activated macrophages.
‘mature macrophage’ refers to a macrophage which expresses mature cell surface markers, preferably CCR2−, CD14+ and 25F9+.

Macrophages may acquire various states, referred to as “polarisation”, which are usually, but simplistically, divided into two main extremes, “pro-inflammatory” (or classically-activated, “M1”, “M1-like”) and “pro-regenerative” (or “pre-restorative”, alternatively-activated, “M2”, “M2-like”, anti-inflammatory or anti-fibrotic). However, macrophages may adopt a state between these extremes which may be “unpolarised”, resting or naïve (M0) or point more towards an anti or pro-inflammatory state.

It is generally postulated that M1 macrophages are pro-inflammatory, whereas M2 macrophages are responsible for immunomodulation and wound-healing responses. However, it is increasingly clear that this binary classification does not address the more complex heterogeneity in vivo, where macrophages adopt distinct phenotypes and even switch between phenotypes in response to the myriad of stimuli to which they are exposed. These in vivo macrophage phenotypes are impossible to recapitulate exactly in tissue culture models, emphasizing the importance of the characterization of macrophages on the basis of function.

Macrophages acquire a “pro-regenerative” state under the action of various factors in combination, including macrophage colony-stimulating factor (M-CSF), IL-4, IL-13, IL-10 and TGF-β. These macrophages mediate wound healing and tissue regeneration primarily.

The engineered macrophages of the present invention may function as “Pro-restorative” macrophages. As described in e.g. Ramachandran et al, Proc Natl Acad Sci USA. 2012 Nov. 13; 109(46): E3186-E3195, pro-restorative macrophages have a loss of pro-inflammatory gene expression, increased expression of matrix-degrading enzymes, and enrichment of phagocytosis-related genes.

Furthermore, the phenotype of pro-restorative macrophages falls outside the M1/M2 paradigm, highlighting the limitations of this classification in an in vivo setting. Pro-restorative macrophages play an important role in tissue remodelling e.g. fibrosis resolution. The engineered macrophages may be exposed to an anti-inflammatory treatment.

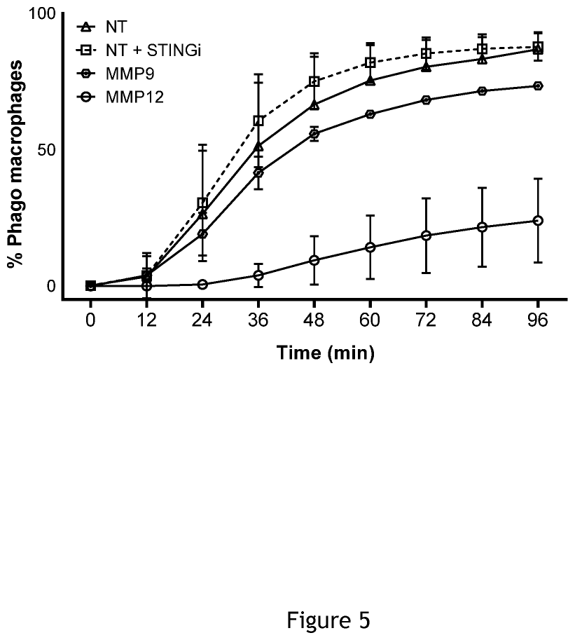

The macrophages reported in Ramachandran et al have high MMP activity. Further, in FIG. 5 the authors show several genes regulated in the pro-restorative macrophages. Of note, they see an upregulation of Mrc1 (now CD206) which is also expressed in the macrophages of the Examples presented here. Further, they show low levels of TNFα and IL1b. Such may therefore be indicative of a pro-restorative phenotype. ‘GMP-compliant’ as used herein means that the method complies with Good Manufacturing Practice principles and may be used interchangeably with ‘GMP-compatible’ and ‘GMP-graded’. By way of example a GMP-compliant medium has to be serum-free, antibiotic-free, animal substance free and xenoprotein-free. The WHO provides guidance on what is required for good manufacturing practice: “Chapter 1: WHO good manufacturing practices: Main principles for pharmaceutical products”. Quality Assurance of Pharmaceuticals: A compendium of guidelines and related materials—Good manufacturing practices and inspection. 2 (2nd updated ed.). WHO Press. pp. 17-18. ISBN 9789241547086.

It is to be noted that the term “a” or “an” entity refers to one or more of that entity.

‘about’ means+/−10% of the value given, +/−9%, +/−8%, +/−7%, +/−6%, +/−5%, +/−4%, +/−3%, +/−2%, +/−1%, unless otherwise stated.

A “therapeutically effective amount” of macrophages described in this specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g. animal model) systems.

Suitably the subject may be in need of treatment. Suitably therefore the subject may have a disease, condition or disorder or be at risk of developing a disease, condition or disorder. Suitably the subject may display one or more symptoms of a disease, condition or disorder.

### EQUIVALENTS

Those skilled in the art will recognise or be able to ascertain using no more than routine experimentation, equivalents of the specific embodiments described herein. Such equivalents are intended to be encompassed by the following claims. Any combination of the embodiments disclosed in the any plurality of the dependent claims or Examples is contemplated to be within the scope of the disclosure.

### INCORPORATION BY REFERENCE

The disclosure of each and every patent, patent application publication, and scientific publication referred to herein is specifically incorporated herein by reference in its entirety, as are the contents of its Figures.

### REFERENCES

1 Williams, R. et al. Addressing liver disease in the UK: a blueprint for attaining excellence in health care and reducing premature mortality from lifestyle issues of excess consumption of alcohol, obesity, and viral hepatitis. Lancet 384, 1953-1997, doi:10.1016/S0140-6736(14)61838-9 (2014).
2 Pellicoro, A., Ramachandran, P., Iredale, J. P. & Fallowfield, J. A. Liver fibrosis and repair: immune regulation of wound healing in a solid organ. Nat Rev Immunol 14, 181-194, doi:10.1038/nri3623 (2014).
3 Fallowfield, J. A. Future mechanistic strategies for tackling fibrosis—an unmet need in liver disease. Clin Med (Lond) 15 Suppl 6, s83-87, doi:10.7861/clinmedicine.15-6-s83 (2015).
4 Forbes, S. J. & Newsome, P. N. Liver regeneration—mechanisms and models to clinical application. Nat Rev Gastroenterol Hepatol, doi:10.1038/nrgastro.2016.97 (2016).
5 Thomas, J. A., Ramachandran, P. & Forbes, S. J. Studies of macrophage therapy for cirrhosis —From mice to men. J Hepatol 68, 1090-1091, doi:10.1016/j.jhep.2017.11.043 (2018).
6 Forbes, S. J., Gupta, S. & Dhawan, A. Cell therapy for liver disease: From liver transplantation to cell factory. J Hepatol 62, S157-169, doi:10.1016/j.jhep.2015.02.040 (2015).
7 Falasca, L., Bergamini, A., Serafino, A., Balabaud, C. & Dini, L. Human Kupffer cell recognition and phagocytosis of apoptotic peripheral blood lymphocytes. Exp Cell Res 224, 152-162, doi:10.1006/excr.1996.0123 (1996).
8 Ramachandran, P. et al. Differential Ly-6C expression identifies the recruited macrophage phenotype, which orchestrates the regression of murine liver fibrosis. Proc Natl Acad Sci USA 109, E3186-3195, doi:10.1073/pnas.1119964109 (2012).
9 Wynn, T. A. & Barron, L. Macrophages: master regulators of inflammation and fibrosis. Semin Liver Dis 30, 245-257, doi:10.1055/s-0030-1255354 (2010).
10 Zigmond, E. et al. Infiltrating monocyte-derived macrophages and resident kupffer cells display different ontogeny and functions in acute liver injury. J Immunol 193, 344-353, doi:10.4049/jimmunol.1400574 (2014).
11 Campana, L., Esser, H., Huch, M. & Forbes, S. Liver regeneration and inflammation: from fundamental science to clinical applications. Nat Rev Mol Cell Biol 22, 608-624, doi:10.1038/s41580-021-00373-7 (2021).
12 Thomas, J. A. et al. Macrophage therapy for murine liver fibrosis recruits host effector cells improving fibrosis, regeneration, and function. Hepatology 53, 2003-2015, doi:10.1002/hep.24315 (2011).
13 Moore, J. K. et al. Phenotypic and functional characterization of macrophages with therapeutic potential generated from human cirrhotic monocytes in a cohort study. Cytotherapy 17, 1604-1616, doi:10.1016/j.jcyt.2015.07.016 (2015).
14 Fraser, A. R. et al. Development, functional characterization and validation of methodology for GMP-compliant manufacture of phagocytic macrophages: A novel cellular therapeutic for liver cirrhosis. Cytotherapy 19, 1113-1124, doi:10.1016/j.jcyt.2017.05.009 (2017).
Iredale, J. P. Tissue inhibitors of metalloproteinases in liver fibrosis. Int J Biochem Cell Biol 29, 43-54, doi:10.1016/s1357-2725(96)00118-5 (1997).
16 Woo, C. H., Lim, J. H. & Kim, J. H. Lipopolysaccharide induces matrix metalloproteinase-9 expression via a mitochondrial reactive oxygen species-p38 kinase-activator protein-1 pathway in Raw 264.7 cells. J Immunol 173, 6973-6980, doi:10.4049/jimmunol.173.11.6973 (2004).
17 Lolmede, K. et al. Inflammatory and alternatively activated human macrophages attract vessel-associated stem cells, relying on separate HMGB1- and MMP-9-dependent pathways. J Leukoc Biol 85, 779-787, doi:10.1189/jlb.0908579 (2009).
18 Liao, Y., Zhu, E. & Zhou, W. Ox-LDL Aggravates the Oxidative Stress and Inflammatory Responses of THP-1 Macrophages by Reducing the Inhibition Effect of miR-491-5p on MMP-9. Front Cardiovasc Med 8, 697236, doi:10.3389/fcvm.2021.697236 (2021).
19 Gratchev, A., Kzhyshkowska, J., Utikal, J. & Goerdt, S. Interleukin-4 and dexamethasone counterregulate extracellular matrix remodelling and phagocytosis in type-2 macrophages. Scand J Immunol 61, 10-17, doi:10.1111/j.0300-9475.2005.01524.x (2005).
20 Giannandrea, M. & Parks, W. C. Diverse functions of matrix metalloproteinases during fibrosis. Dis Model Mech 7, 193-203, doi:10.1242/dmm.012062 (2014).
21 Cabrera, S. et al. Overexpression of MMP9 in macrophages attenuates pulmonary fibrosis induced by bleomycin. IntJ Biochem Cell Biol 39, 2324-2338, doi:10.1016/j.biocel.2007.06.022 (2007).
22 Wang, Q. et al. Dynamic features of liver fibrogenesis and fibrosis resolution in the absence of matrix metalloproteinase9. Mol Med Rep 20, 5239-5248, doi:10.3892/mmr.2019.10740 (2019).
23 Campana, L. & Iredale, J. P. Regression of Liver Fibrosis. Semin Liver Dis 37, 1-10, doi:10.1055/s-0036-1597816 (2017).
24 Zhao, H. et al. Matrix metalloproteinases contribute to kidney fibrosis in chronic kidney diseases. World J Nephrol 2, 84-89, doi:10.5527/wjn.v2.i3.84 (2013). Maretti-Mira, A. C., de Pinho Rodrigues, K. M., de Oliveira-Neto, M. P., Pirmez, C. & Craft, N.
25 MMP-9 activity is induced by Leishmania braziliensis infection and correlates with mucosal leishmaniasis. Acta Trop 119, 160-164, doi:10.1016/j.actatropica.2011.05.009 (2011).
26 Afratis, N. A., Selman, M., Pardo, A. & Sagi, I. Emerging insights into the role of matrix metalloproteases as therapeutic targets in fibrosis. Matrix Biol 68-69, 167-179, doi:10.1016/j.matbio.2018.02.007 (2018).
27 Iredale, J. P. A cut above the rest?MMP-8 and liver fibrosis gene therapy. Gastroenterology 126, 1199-1201, doi:10.1053/j.gastro.2004.01.060 (2004).
28 Rojkind, M., Giambrone, M. A. & Biempica, L. Collagen types in normal and cirrhotic liver. Gastroenterology 76, 710-719 (1979).
29 Bearzi, C. et al. PIGF-MMP9-engineered iPS cells supported on a PEG-fibrinogen hydrogel scaffold possess an enhanced capacity to repair damaged myocardium. Cell Death Dis 5, e1053, doi:10.1038/cddis.2014.12 (2014).
Laurenzana, A. et al. Melanoma cell therapy: Endothelial progenitor cells as shuttle of the MMP12 uPAR-degrading enzyme. Oncotarget5, 3711-3727, doi:10.18632/oncotarget.1987 (2014).
31. Trebicka J, et al. International Variceal Bleeding Observational Study Group and Baveno Cooperation. Rebleeding and mortality risk are increased by ACLF but reduced by pre-emptive TIPS. J Hepatol 73(5):1082-1091 (2020)
32. Moreau R, et al. CANONIC Study Investigators of the EASL-CLIF Consortium. Acute-on-chronic liver failure is a distinct syndrome that develops in patients with acute decompensation of cirrhosis. Gastroenterology, 144(7):1426-37 1437.e1-9. (2013)
33. Starkey Lewis P J, Moroni F, Forbes S J. Macrophages as a Cell-Based Therapy for Liver Disease. Semin Liver Dis., 39(4):442-451. (2019)
34. Cabrera S, Gaxiola M, Arreola J L, Ramirez R, Jara P, D'Armiento J, Richards T, Selman M, Pardo A. Overexpression of MMP9 in macrophages attenuates pulmonary fibrosis induced by bleomycin. Int J Biochem Cell Biol. 2007; 39(12):2324-38.

### Examples

### Materials and Methods

hMDMs Cell Culture

Monocytes were isolated from a buffy coat product from a healthy volunteer sourced from the Scottish National Blood Transfusion Service (SNBTS) using a Ficoll gradient (GE Healthcare) followed by a magnetic column selection using CliniMACS CD14 Reagent (Miltenyi Biotec). Monocytes were matured for 1 to 7 days in culture in TexMACS without phenol red (Miltenyi Biotec) in the presence of 100 ng/mL GMP-graded recombinant human macrophage colony-stimulating factor (rhM-CSF) (R&D System, Biotechne). hMDMs are cultured in 6 wells multi-well plate (Corning Costar) at a density of 2×10⁶/cm² for five days. hMDMs were counted using an automated counter (TC20, BioRad).

### Payload Transfection

Day 5 human monocyte-derived macrophages (hMDMs) were resuspended in CliniMACS® Electroporation Buffer (Miltenyi Biotec, #170-076-625) at density 75*10\hat{ }6 or 150*10\hat{ }6 cells/ml and 100-300 μl suspension was transferred to an electroporation cuvette with 0.2 cm gap size. 5 μg plasmid DNA per 5*10\hat{ }6 cells were added directly into the cuvette and mixed with the cells by gently flicking the cuvette. The cells were transfected using CliniMACS® Electroporator controlled by CliniMACS Prodigy®. The parameters for electroporation are outlined in Table 2 below (Patent PCT/GB2021/051300):

TABLE 2
Parameters for transfection by electroporation of MMP-expressing plasmid in hMDMs.

| 1st pulse | | | | | 2nd pulse | | | |
|---|---|---|---|---|---|---|---|---|
| Voltage | Length | | | Burst length | Voltage | Length | | Burst length |
| (V) | (μsec) | Mode | Polarity | (μsec) | (V) | (μsec) | Mode | (μsec) |
| 950 | 120 | Burst | Unipolar | 20 | 100 | 23000 | Burst | 5 |

Following transfection, the cells were recovered from the cuvette into sterile TexMACS™ GMP medium (Miltenyi Biotec, #170-076-306) using a 18G sterile needle attached to a 1 mL syringe or similar tools. Cell count was performed using TC-20 automated cell counter (Bio-Rad). The cells were spun down at 300*g, 5 min, at room temperature. The supernatant was aspirated, and the cells were resuspended in sterile TexMACS™ GMP medium (Miltenyi Biotec, #170-076-306) supplemented with 100 ng/mL rhM-CSF (R&D systems, #AFL216), 20 ng/mL rhIL4 (R&D systems, #AFL204) and 20 ng/mL rhIL13 (R&D systems, #213-ILB/CF) at concentration 4*10\hat{ }6 cells/mL and plated at density 2*10\hat{ }6 cells/cm²

### Flow Cytometry Labelling

Macrophages were resuspended at a concentration of 1×10⁶/ml in PBS+0.5 mM EDTA (Life Technologies)+FcR Block 1:100 (Miltenyi). Aliquoted 100 ul of cells into low adherence, round bottomed 96 well plates. Incubated cells for 5 minutes, then added appropriate antibodies (see Table 3) to appropriate test wells and left for 20 min at 4 C. Washed cells with PBS+0.5 mM EDTA and spun at 300 g for 5 min. Removed the supernatants and resuspended cells in PBS+0.5 mM EDTA+1:1000 DRAQ7. Incubated for 5 min at 4 C. Washed as before, then resuspended in 100 ul of PBS+0.5 mM EDTA+0.1% human serum. Acquired 50 ul of cells on the Novocyte3000 or Novocyte Quanteon (Agilent).

TABLE 3
Antibodies used for flow cytometry labelling of hMDMs.

| ANTIGEN | FLUOROPHORE | CLONE | SUPPLIER | ORDERING CODE |
|---|---|---|---|---|
| CD45 | VB | 5B1 | Miltenyi Biotec | 130-092-880 |
| CD14 | VB/PE | TUK4 | Miltenyi Biotec | 130-091-242/130-113-152 |
| CD206 | FITC | | Miltenyi Biotec | 130-095-131 |
| 25F9 | APC | 25F9 | eBioscience | 50-0115-42 |
| CCR2 | PE | K036C2 | BioLegend | 357206 |
| CD163 | FITC | | Miltenyi Biotec | BO-097-626 |
| CD169 | APC | 7-239 | BioLegend | 346008 |
| CD86 | PE | BU63 | BioLegend | 374206 |
| MHC II | FITC | TU39 | BioLegend | 361705 |

### MSD V-plex Cytokine Dosage

Cytokines in cell culture supernatants were analysed using a V-PLEX Human Biomarker 10-Plex kit on a MESO Quickplex SQ 120 according to the manufacturers' instructions (Meso Scale Discovery). 10 uL of supernatants were tested. Results are in pg/mL. Values are adjusted taking into consideration the dilution at the time of testing. All data shown represent the secretion in a 24 h period. Data reported are net concentration, as calculated by subtracting the amount of the given cytokine in culture medium alone (TexMACS) to the amount of cytokine detected in the cell culture supernatants.

RNA Extraction and gPCR

RNA was extracted using RNeasy Mini Kit (Qiagen) according to manufacturer's instruction. One-step real time qPCR was conducted using the extracted RNA with the QuantiTect SYBR Green RT PCR Kit and QuantiTech Primers (Qiagen). Briefly, master mix was prepared, RNA was diluted to 5 ng/μL and 1 uL was added per test to the master mix. One step qPCR was conducted in a 384-well Quant studio 5 (ThermoFisher) on a program of 50° C. for 10 min, then 95° C. for 15 min for reverse transcription and cDNA synthesis, followed by 40 cycles of 94° C. for 15 sec, 55° C. for 30 sec and 72° C. for 30 sec. PIK3C2A and HPRT1 were the two housekeeping genes used for normalisation and RNA expression was calculated using the 2^{-ΔCt} method (ΔCt=Ct (gene of interest) ˜Ct (GeoMean of 2 housekeeping genes)).

### MMP Activity Assay

MMP activity was confirmed via successful cleavage of a standard MMP peptide. The standard MMP peptides are flanked with a quencher and fluorescent signal, which when intact, do not fluoresce. Cleaved peptides no longer quench the fluorescent signal, therefore resulting in an emission of fluorescence which is measured as relative fluorescent units (RFU). The assay was carried out in accordance with manufacturer's instructions ttps://www.abcam.com/ps/products/112/ab112146/documents/ab112146%20 MMP %20Activity %2 OAssay %20Kit %20Fluorometric %20-%20Green %20v4b %20(website).pdf (ab112146 MMP Activity Assay Kit Fluorometric—Green v4b (website).pdf (abcam.com)). 25 ul of cell culture supernatants were tested. All data shown represent the activity of MMPs secreted over a 24-hour period. Results are plotted as RFU minus background fluorescence of culture medium alone (TexMACS).

### MSD R-Plex MMP Dosage

MMPs in cell culture supernatants were analysed using an R-PLEX Human Biomarker 7-Plex kit on a MESO Quickplex SQ 120 according to the manufacturers' instructions (Meso Scale Discovery. 15 μL of supernatants were tested. Results are in pg/mL. Values are adjusted taking into consideration the dilution at the time of testing. All data shown represent the secretion in a 24 h period. Data reported are net concentration, as calculated by subtracting the amount of the given cytokine in culture medium alone (TexMACS) to the amount of cytokine detected in the cell culture supernatants.

### Phagocytosis—Live Imaging

hMDMs were plated at a density of 250,000 cells/well in a 96-well clear bottom imaging plate (Grenier) and left to adhere for 24 hours at 37 degrees Celsius at 5% CO₂ in 100 ul of TexMACS+MCSF (non-transfected hMDMs) or TexMACS+MCSF+IL4/IL13 (STING inhibition cocktail, transfected hMDMs). Supernatant was removed and cells were stained for 30 mins with 100 ul PBS+NucBlue (ThermoFisher)+5 μg/ml Cellmask Deep red plasma membrane stain (Invitrogen) at 37 degrees Celsius, 5% CO₂. Cells were washed three times with 100 μl PBS. 50 ul of PBS was added to cells for TO analysis on the Opera Phenix High Content Screening System. 50 μl of 0.2 mg/ml pHrodo Red Zymosan beads (Life Technologies) were added to cells after TO. A series of images were acquired over a period of 96 minutes to monitor phagocytosis. Images were analysed with Columbus data imaging software and Tibco Spotfire data analysis system. Graphs plotted using GraphPad Prism 9.2.0.

### Statistics

Every reported dot is a distinct donor. At least 3 donors were analysed in each condition unless otherwise specified. Where appropriate, data are shown as mean±SD. Where appropriate, two tail t-test for paired data was carried out.

### Results

hMDMs can be Transfected Efficiently with an MMP-Encoding Plasmid

An ideal therapeutic macrophage to be used in treating inflammatory organ damage with a fibrotic component should have anti-fibrotic properties. Non-transfected, non-polarised macrophages express little to no MMPs, therefore hMDMs were transfected with either MMP9 or MMP12. Expression of MMP9 and MMP12 was verified 24h post-transfection. A significant increase in the level of MMP9 and MMP12 mRNA was noted in transfected vs non-transfected hMDMs (FIG. 1A-B). Furthermore, the expression of MMP9 and MMP12 proteins was also significantly increased as compared to non-transfected hMDMs (FIG. 1C-D).

MMP-Transfected hMDMs Secrete Active and Functional MMPs in the Extracellular Space.

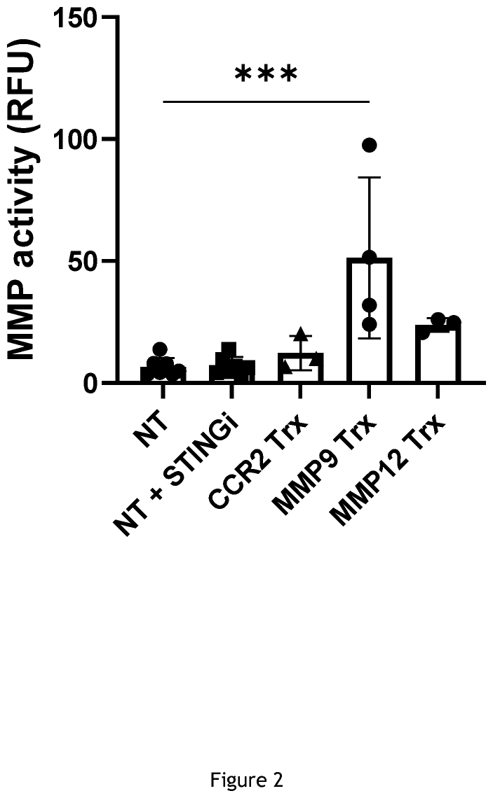

When a cell overexpresses MMPs it can upregulate their inhibitors, the Tissue Inhibitors of Metalloproteinases (TIMPs). The upregulation of TIMPs would make the MMPs non-functional. An enzymatic assay was deployed to verify MMP functionality after secretion in the cell culture medium of non-transfected (NT), non-transfected treated with the STING inhibition cocktail (NT+STINGi) MMP9-transfected (MMP9 Trx), MMP12-transfected (MMP12 Trx), and CCR2-transfected (CCR2 Trx) hMDMs. The results show that MMP9-transfected hMDMs have significantly higher MMP activity in their cell culture supernatants, and MMP12-transfected show a trend towards an upregulation in MMP activity (FIG. 2). The lack of a significant increase of MMP activity in the cell culture supernatants of the CCR2-transfected hMDMs confirms that the increase activity is a specific effect of the transfected payloads and not a general side effect of transfection.

The increased MMP activity in an MMP-transfected macrophage is remarkable: oftentimes, when MMPs are upregulated, Tissue Inhibitors of MMPs (TIMPs) are upregulated, too, thereby limiting the effectiveness of the MMP extracellular activity¹⁵. Therefore, the results suggest that the utilised transfection methods achieve increased secretion of active MMPs, without significant up-regulation of TIMPs.

Taken together, these data support the use of MMP-transfected hMDMs in inflammatory diseases with a fibrotic component. MMPs may be transfected alone or in combination.

MMP-Transfected hMDMs Maintain Expression of Macrophage-Specific Markers

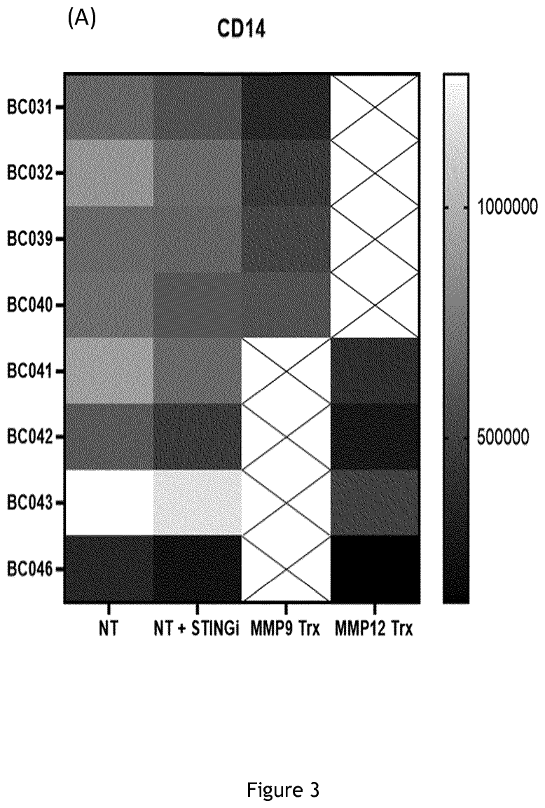

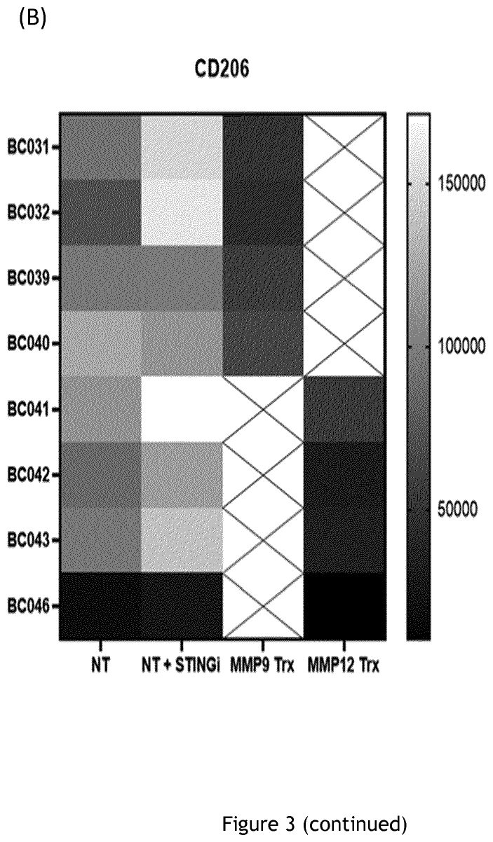

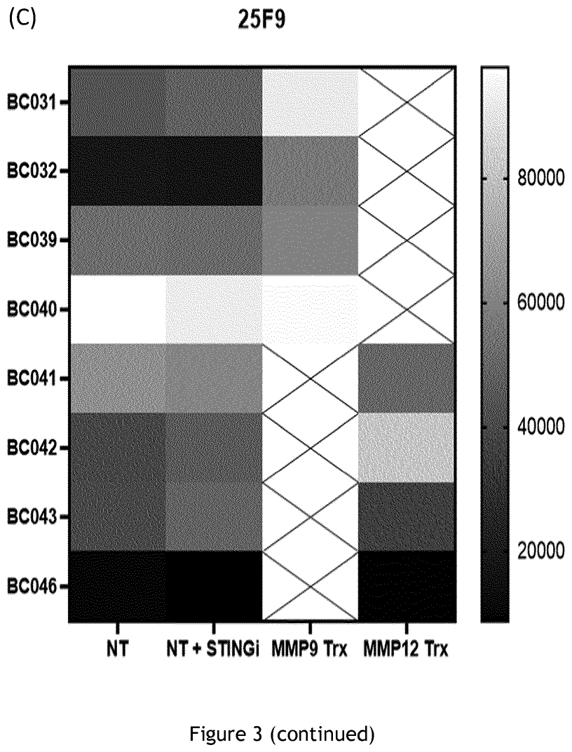

To demonstrate that MMP-transfected hMDMs have a favourable safety profile, the expression of markers associated with macrophage lineage and function were measured. A full report of the average MFI detected in non-transfected hMDMs (NT), NT treated with the STING inhibitors cocktail (STINGi), MMP9-transfected (MMP9 Trx) and MMP12 Trx hMDMs can be found in Table 4. FIG. 3 is a graphic representation of the data in Table 4.

TABLE 4
Average MFI of cell surface markers expression
measured by flow cytometry in four
donors/Trx group, and eight donors in control
groups (NT and NT + STINGi).

| Markers | NT | NT + STINGi | MMP9 Trx | MMP12 Trx |
|---|---|---|---|---|
| CD14 | 769398 | 641760 | 486791 | 331065 |
| CD206 | 86911 | 121637 | 54471 | 32769 |
| 25F9 | 45755 | 46850 | 76252 | 44102 |

FIG. 3 shows that there is a modulation of some markers, such as CD206, whilst other are stable, such as 25F9. Overall, the expression of macrophage lineage and function marker is still detectable by flow cytometry, thereby confirming that transfection has no significant effects on cell identity.

MMP9 Transfection Modifies hMDMs Inflammatory and Anti-Fibrotic Secretion Profile

Other key features of an anti-fibrotic cell therapy are its inflammatory profile and its ability to secrete MMPs other than the specific overexpressed MMP⁸.

Both features were investigated using a multi-protein dosage system. To evaluate the inflammatory profile CCR2 Trx hMDMs have been used as a control, to discriminate the payload-specific effect vs. the effect of the transfection procedure. MMP9-Trx and MMP12-Trx hMDMs upregulate pro-inflammatory cytokines, such as TNFα, IL12p70 and IL8 (FIG. 4A and Table 5). This is only partially surprising: it was previously reported that pro-inflammatory macrophages, stimulated with IFNγ and LPS, upregulate MMP9¹⁶. However, previous research also found an upregulation of MMP9 in human pro-restorative macrophages, following in vitro stimulation with IL10⁷. Furthermore, studies conducted using mouse models of chronic liver injury have identified a population with a hybrid pro-inflammatory/pro-restorative phenotype, which is involved in liver regeneration, and overexpresses MMP9 and MMP12 but not pro-inflammatory mediators such as TNFα or IL12p70⁸. Finally, a direct correlation between MMP9 overexpression and inflammatory phenotype has been described in THP-1 cells18.

To evaluate the MMP secretion profile a multi-protein dosage method that allows for the simultaneous evaluation of MMP1, 3, 7, 8 and 10 was used. Conditioned media from non-transfected (NT), non-transfected+STING inhibitor cocktail (NT+STINGi) and MMP-transfected hMDMs were analysed. It was previously demonstrated that IL4 suppresses the expression of MMPs¹⁹. IL4 is part of the STING inhibition cocktail, together with IL13. Therefore, it was possible that the use of STINGi repressed the expression of one or more MMPs, including MMP9 itself. Surprisingly, MMPTrx hMDMs strongly upregulate all the MMPs dosed, beside MMP9 itself (FIG. 4B). Thus, the genetic engineering method presented herein is suitable for the generation of a cell therapy with anti-fibrotic properties.

TABLE 5
Average/analyte/group utilised to generate the heat map in FIG. 4A.

| | NT | NT + STINGi | CCR2 Trx | MMP9 Trx | MMP12 Trx |
|---|---|---|---|---|---|
| IL10 | 1.06 | 0.77 | 682.28 | 776.66 | 264.17 |
| IL12p70 | 0.46 | 0.62 | 0.88 | 62.02 | 13.33 |
| IL1ß | 0.58 | 0.29 | 2.76 | 24.21 | 17.73 |
| IL2 | 0.35 | 0.31 | 0.58 | 8.75 | 8.50 |
| IL6 | 2.23 | 0.69 | 28.28 | 977.44 | 440.19 |
| IL8 | 538.58 | 245.41 | 1171.57 | 7756.63 | 7691.04 |
| IFNY | 1.93 | 0.39 | 32.25 | 75.88 | 134.28 |
| TNFa | 3.14 | 1.51 | 80.49 | 2501.92 | 2832.72 |

Average of at least n = 4/group.

MMP9, but not MMP12-Transfected hMDMs have Similar Phagocytosis than Non-Transfected Macrophages

The last step in the characterisation of MMP-transfected macrophages is the measurement of their phagocytic capacity. MMP9 Trx, MMP12, not-transfected (NT) hMDMs and NT treated with the STING inhibition cocktail (STINGi) were fed with E. coli-coated, pH-sensitive beads for 1.5h. Phagocytosis was measured by live imaging.

Data in FIG. 5 show that MMP12-transfected hMDMs have lower phagocytic capacity as compared to NT and NT+STINGi hMDMs. However, MMP9-transfected hMDMs show similar phagocytosis to NT and NT+STINGi hMDMs.

These data suggest that careful consideration is needed when choosing the appropriate MMP to use as therapeutic strategy. Furthermore, the data support the use of distinct MMPs in combination so to maximise the variety of targets in the fibrotic extracellular matrix, whilst maintaining other functions such as phagocytosis.

### CONCLUSIONS AND DISCUSSION

MMPs have a complex biology, which often is organ and disease specific²⁰. For example, MMP9 has a marked anti-fibrotic effect in models of chronic liver disease and chronic lung disease²¹⁻²³. However, it seems to be detrimental in kidney fibrosis²⁴. The results presented herein support the use of a DNA vector expressing a specific MMP of choice to genetically modify macrophages, allowing for tailoring of the anti-fibrotic cell therapy to the organ and condition of interest.

In pathophysiology, macrophages are some of the bigger expressors of MMPs. However, as shown in FIGS. 1 and 4, in vitro derived hMDMs express negligible levels of MMPs; plus, hardly any activity is detected in the conditioned media of these cells (FIG. 2). In models of chronic liver disease, it has been demonstrated that a macrophage with a hybrid phenotype but pro-restorative function exists, and is characterised by expression of MMP9 and MMP12⁸. It is though very difficult to replicate this phenotype in vitro: in FIG. 4B The inventors show that the IL4+1L13 STING inhibition cocktail promotes expression of a different set of MMPs as compared to non-transfected, non-treated macrophages. However, both cell types have negligible MMP activity detected in their supernatants (FIG. 2), thereby making them unsuitable as prominent anti-fibrotic agents. MMPs are normally up regulated upon infection²⁵, or in vitro stimulation with LPS¹⁶. Stimulating hMDMs with LPS is not a viable option for cell therapy, therefore a different strategy to upregulate MMPs must be sought. In the present invention, specific MMPs of interest were introduced to macrophages by transfection. As proof of principle, day5 hMDMs were transfected with plasmids expressing MMP9 (a type IV collagenase) and MMP12 (an elastase), two MMPs having distinct target proteins in the extracellular matrix. As shown in FIG. 1 marked overexpression of both MMP9 and MMP12 is achieved with this method. Furthermore, significant increase in the MMP activity was detected in the conditioned media from the transfected hMDMs, when compared to the non-transfected hMDMs media (FIG. 2). A plasmid expressing CCR2 was used as a control, to show that the upregulation in MMP activity in the conditioned media is payload-specific and is not simply the result of the transfection procedure. The increase in the MMP activity in transfected cells is not obvious for two reasons: (i) RNA expression of MMPs does not necessarily translate into secretion into the extracellular space of an active form of the enzyme^{20,23,26}. However the approach outlined herein achieves proficient secretion of active MMPs. (ii) It is known that when MMPs are upregulated, their tissue inhibitors (TIMPs) are upregulated, too, in a negative feedback loop^{15,23}. Achieving a sustained increase in MMP activity by the herein described methods therefore demonstrates that this technology achieves upregulation of functional MMPs, surprisingly without the upregulation of TIMPs.

The most surprising feature of the MMP-transfected macrophages of the present invention is their ability to co-upregulate other MMPs beyond the exogenously-encoded MMP: FIG. 4B shows that MMP9 Trx hMDMs strongly upregulate several other MMPs, such as MMP1, 8 and 10, which could contribute significantly to the anti-fibrotic effect of the macrophage cell therapy of the present invention. For example, MMP8 is normally contained in neutrophil granules and digests collagen I, II, and III, thereby of particular interest for liver fibrosis²⁷, where most of the collagen in the fibrotic septa is collagen I and III²⁸.

MMP-transfected macrophages maintain cell surface marker expression of macrophage-specific markers such as CD14, CD206 and 25F9 (FIG. 3), thereby demonstrating that transfection with MMPs does not alter their cell identity. However, interestingly, MMP9 transfected cells are much better phagocytes as compared to MMP12 transfected cells (FIG. 5). This further demonstrates the versatility of the therapeutic approach described herein, as the appropriate MMP with the appropriate combination of anti-fibrotic and pro-phagocytic capacity can be chosen depending on the disease target.

MMP-transfected macrophages show a more pro-inflammatory profile as compared to non-transfected and CCR2-transfected hMDMs. Therefore, in case the anti-fibrotic therapy is used in the context of a chronic inflammatory disease, a further line of intervention to lower inflammation in MMP-transfected hMDMs may be required. Suitable anti-inflammatory treatments may include treatment with IL-4/1l-13 or the like.

Thus, the present invention relates to a novel approach to anti-fibrotic cell therapies by means of transfecting macrophages to overexpress one or more metalloproteinases selected from MMP9 and MMP12. The type and combination of metalloproteinase can be tailored depending on the fibrotic disease targeted.

1. An engineered macrophage for use in treating an inflammatory condition with a fibrotic element, wherein the macrophage is engineered with exogenous nucleic acid encoding MMP9.

2. An engineered macrophage as claimed in claim 1, wherein said macrophage is derived from a precursor cell.

3. An engineered macrophage according to claim 1 or 2, wherein the inflammatory condition is in the lung or in the liver, preferably in the liver, more preferably wherein the inflammatory condition comprises liver cirrhosis or ACLF.

4. An engineered macrophage according to claim 3 wherein the inflammatory condition comprises liver cirrhosis.

5. An engineered macrophage according to claim 4 wherein the liver cirrhosis resulted from at least one disease or condition selected from the group consisting of: non-alcoholic fatty liver disease (NAFL) (e.g., non-alcoholic fatty liver disease (NAFLD) or non-alcoholic steatohepatitis (NASH)), alcoholic liver disease (e.g., alcoholic fatty liver disease (AFLD) or alcoholic steatohepatitis (ASH)), mechanical trauma to the liver, biliary obstruction, autoimmune hepatitis, iron overload, Hepatitis B infection (HBV) and Hepatitis C infection (HCV).

6. An engineered macrophage according to claim 4 wherein the liver cirrhosis resulted from steatotic liver disease (SLD), optionally wherein the steatotic liver disease is metabolic dysfunction-associated steatotic liver disease (MASLD), metabolic-associated steatohepatitis (MASH), Met-ALD or Cryptogenic SLD.

7. An engineered macrophage according to any of claims 4-6, wherein the liver cirrhosis is selected from compensated cirrhosis and decompensated cirrhosis.

8. An engineered macrophage according to claim 7, wherein the cirrhosis is decompensated cirrhosis, and the subject to be treated exhibits one or more clinical signs of decompensated cirrhosis selected from the list consisting of jaundice, ascites, hepatic encephalopathy, hepatorenal syndrome, variceal haemorrhage and gastrointestinal haemorrhage.

9. An engineered macrophage as claimed in any preceding claim, wherein said macrophage overexpresses the said coding sequence encoding MMP9.

10. An engineered macrophage as claimed in any preceding claim, wherein said exogenous nucleic acid comprises a DNA molecule, an RNA molecule, or a non-viral vector.

11. An engineered macrophage as claimed in any preceding claim, wherein said exogenous nucleic acid is transfected into the macrophage, preferably via electroporation.

12. An engineered macrophage as claimed in any preceding claim wherein said macrophage has a repressed STING pathway or is treated with anti-inflammatory agents.

13. An engineered macrophage according to any preceding claim, wherein said macrophage exhibits a pro-restorative phenotype.

14. An engineered macrophage according to any preceding claim, wherein said macrophage is autologous or allogenic to the subject, optionally wherein the macrophage is derived from a human monocyte, or a stem cell, further optionally wherein the stem cell is an induced pluripotent stem cell.

15. An engineered macrophage according to any preceding claim, wherein said inflammatory condition is chronic.

16. An engineered macrophages according to any preceding claim, wherein the engineered macrophage secretes MMP9 and the secreted MMP9 protein level is greater than 200 ng/ml when the macrophage is cultured in vitro at a cell concentration of 4×10⁶/ml.

17. A population of engineered macrophages comprising engineered macrophages for use according to any preceding claim.

18. A composition comprising an engineered macrophage or population of engineered macrophages for use according to any preceding claim.

19. A method of improving the resolution of fibrosis in a chronic condition, comprising the use of an engineered macrophage, population of engineered macrophages or a composition according to any of the preceding claims.

20. A method of treating an inflammatory condition with a fibrotic element, comprising the use of a macrophage engineered with an exogenous sequence encoding for MMP9.

21. An engineered macrophage, wherein the macrophage is engineered to express MMP9.

22. The engineered macrophage of claim 21, wherein the engineered macrophage comprises one or more exogenous coding sequences for MMP9.

23. An engineered macrophage of claim 22, wherein said exogenous nucleic acid is transfected into the macrophage, optionally via electroporation.

24. The engineered macrophage of claim 22 or 23, wherein the macrophage is transfected with a DNA vector, an RNA vector optionally mRNA, a vector not derived from a viral genome or one or more free nucleic acids.

25. The engineered macrophage of any of claims 21-24, wherein the MMP9 protein expressed is at least 85%, at least 90%, at least 95%, 96%, 97%, 98%, 99% homologous or similar to the sequence presented as SEQ ID No. 2.

26. The engineered macrophage of any one of claims 21-25, wherein the sequence of the MMP9 coding sequence provided is at least 85%, at least 90%, at least 95%, 96%, 97%, 98%, 99% homologous or similar to SEQ ID NO: 1.

27. The engineered macrophage of any one of claims 21-25, wherein the sequence of the MMP9 coding sequence provided is at least 80%, at least 85%, at least 90%, at least 95%, at least 96%, at least 97%, at least 98%, or at least 99% identical to SEQ ID No. 5.

28. The engineered macrophage of claim 21, wherein the macrophage is engineered to turn on or upregulate endogenous genes that encode MMP9.

29. The engineered macrophage of claim 21 or 28, wherein the macrophage is engineered to turn off or downregulate endogenous genes which downregulate MMP9 expression.

30. The engineered macrophage of any one of claims 21-29, wherein the macrophage is engineered to overexpress MMP9.

31. The engineered macrophage of any one of claims 21-30, wherein the macrophage is derived from a human monocyte or a stem cell, optionally an induced pluripotent stem cell.

32. An engineered macrophage of any of claims 21-31, wherein said macrophage has a repressed STING pathway or is treated with anti-inflammatory agents.

33. An engineered macrophage of any of claims 21-32, wherein said macrophage exhibits a pro-restorative phenotype.

34. An engineered macrophage of any of claims 21-33, wherein the engineered macrophage secretes MMP9 and the secreted MMP9 protein level is greater than 200 ng/ml when cultured in vitro at a cell concentration of 4×10⁶/ml.

35. A population of engineered macrophages comprising engineered macrophages according to any of claims 21-34.

36. A composition comprising an engineered macrophage of any of claims 21-34, or population of engineered macrophages according to claim 35.

37. An engineered macrophage, population of engineered macrophages or a composition according to any of claims 21-36 for use in treating an inflammatory condition, preferably an inflammatory condition with a fibrotic element, preferably liver cirrhosis.

38. A method of improving the resolution of fibrosis in a chronic condition, comprising the use of an engineered macrophage, population of engineered macrophages or a composition according to any of claims 21-36.
